SEQUENCE LISTING (62 sequences)

SEQ ID NO: 1 - DNA, 25 bp, Artificial Sequence
  Description of Artificial Sequence Synthetic primer
gagaacaatgagaaccttcaggaga

SEQ ID NO: 2 - DNA, 23 bp, Artificial Sequence
  Description of Artificial Sequence Synthetic primer
ttctggcgccggttacagaacca

SEQ ID NO: 3 - DNA, 25 bp, Artificial Sequence
  Description of Artificial Sequence Synthetic primer
accacttccctcatctctcctccaa

SEQ ID NO: 4 - DNA, 25 bp, Artificial Sequence
  Description of Artificial Sequence Synthetic primer
agggtgagggaggcagagacagaaa

SEQ ID NO: 5 - DNA, 22 bp, Artificial Sequence
  Description of Artificial Sequence Synthetic primer
tgcaggaccaaggagactatgt

SEQ ID NO: 6 - DNA, 23 bp, Artificial Sequence
  Description of Artificial Sequence Synthetic primer
taggatgatgacaagaagtagcc

SEQ ID NO: 7 - DNA, 27 bp, Artificial Sequence
  Description of Artificial Sequence Synthetic primer
atcccatttgcaaagcttctggctggc

SEQ ID NO: 8 - DNA, 27 bp, Artificial Sequence
  Description of Artificial Sequence Synthetic primer
tgtgaagcgtctcacaggtccaggatg

SEQ ID NO: 9 - DNA, 25 bp, Artificial Sequence
  Description of Artificial Sequence Synthetic primer
aggtcagcagcatcgtggtcaacat

SEQ ID NO: 10 - DNA, 25 bp, Artificial Sequence
  Description of Artificial Sequence Synthetic primer
gtggggttgtctttgaataccgcag

SEQ ID NO: 11 - DNA, 22 bp, Artificial Sequence
  Description of Artificial Sequence Synthetic primer
ctctgcatcctcaccatcacag

SEQ ID NO: 12 - DNA, 22 bp, Artificial Sequence
  Description of Artificial Sequence Synthetic primer
tagccgtagatgtgcagcgtgt

SEQ ID NO: 13 - DNA, 23 bp, Artificial Sequence
  Description of Artificial Sequence Synthetic primer
taaacacctgcccatctactcgg

SEQ ID NO: 14 - DNA, 23 bp, Artificial Sequence
  Description of Artificial Sequence Synthetic primer
atctcatcatcctgggctgctgg

SEQ ID NO: 15 - DNA, 22 bp, Artificial Sequence
  Description of Artificial Sequence Synthetic primer
cggctggtggagtggatcatag

SEQ ID NO: 16 - DNA, 22 bp, Artificial Sequence
  Description of Artificial Sequence Synthetic primer
ccctctgttgctgcccatctga

SEQ ID NO: 17 - DNA, 18 bp, Artificial Sequence
  Description of Artificial Sequence Synthetic primer
gccttaccacatccgctc

SEQ ID NO: 18 - DNA, 22 bp, Artificial Sequence
  Description of Artificial Sequence Synthetic primer
tcacactcttccgtcacattgc

SEQ ID NO: 19 - DNA, 22 bp, Artificial Sequence
  Description of Artificial Sequence Synthetic primer
agacatcgcactgactgagaac

SEQ ID NO: 20 - DNA, 22 bp, Artificial Sequence
  Description of Artificial Sequence Synthetic primer
gacgggtcactatctgtgcaac

SEQ ID NO: 21 - DNA, 20 bp, Artificial Sequence
  Description of Artificial Sequence Synthetic primer
cttcaagccagaggcctacg

SEQ ID NO: 22 - DNA, 20 bp, Artificial Sequence
  Description of Artificial Sequence Synthetic primer
ccgcctctgtcttcttcagc

SEQ ID NO: 23 - DNA, 24 bp, Artificial Sequence
  Description of Artificial Sequence Synthetic primer
tgcagccaaagtgaagagggaaga

SEQ ID NO: 24 - DNA, 24 bp, Artificial Sequence
  Description of Artificial Sequence Synthetic primer
catagcgagcagcccaaagaagaa

SEQ ID NO: 25 - DNA, 24 bp, Artificial Sequence
  Description of Artificial Sequence Synthetic primer
tgcttgaatgtgctgatgacaggg

SEQ ID NO: 26 - DNA, 25 bp, Artificial Sequence
  Description of Artificial Sequence Synthetic primer
aaggcaagtcagcaggcatctcatc

SEQ ID NO: 27 - DNA, 25 bp, Artificial Sequence
  Description of Artificial Sequence Synthetic primer
gtactggtctcagcagattgaggag

SEQ ID NO: 28 - DNA, 25 bp, Artificial Sequence
  Description of Artificial Sequence Synthetic primer
gcttctgctggcttaatgcctcaga

SEQ ID NO: 29 - DNA, 20 bp, Artificial Sequence
  Description of Artificial Sequence Synthetic primer
atggccgagcagaaccggaa

SEQ ID NO: 30 - DNA, 20 bp, Artificial Sequence
  Description of Artificial Sequence Synthetic primer
ccatgagccgctggtactcc

SEQ ID NO: 31 - DNA, 20 bp, Artificial Sequence
  Description of Artificial Sequence Synthetic primer
tcatcgctcaggaggtcctt

SEQ ID NO: 32 - DNA, 21 bp, Artificial Sequence
  Description of Artificial Sequence Synthetic primer
ctgttgccagagatggaggtt

SEQ ID NO: 33 - DNA, 20 bp, Artificial Sequence
  Description of Artificial Sequence Synthetic primer
gaagactcgcatccgaatgg

SEQ ID NO: 34 - DNA, 22 bp, Artificial Sequence
  Description of Artificial Sequence Synthetic primer
cgcaggataggaggaagagact

SEQ ID NO: 35 - DNA, 20 bp, Artificial Sequence
  Description of Artificial Sequence Synthetic primer
ttagctgaattcgcgtgtgg

SEQ ID NO: 36 - DNA, 22 bp, Artificial Sequence
  Description of Artificial Sequence Synthetic primer
gaggaagtgaatgagccggtta

SEQ ID NO: 37 - DNA, 22 bp, Artificial Sequence
  Description of Artificial Sequence Synthetic primer
gcgccatatccaacagtgacag

SEQ ID NO: 38 - DNA, 22 bp, Artificial Sequence
  Description of Artificial Sequence Synthetic primer
gccagcagttgcattgacataa

SEQ ID NO: 39 - DNA, 22 bp, Artificial Sequence
  Description of Artificial Sequence Synthetic primer
gtaaaagcaaagacgggactgg

SEQ ID NO: 40 - DNA, 22 bp, Artificial Sequence
  Description of Artificial Sequence Synthetic primer
atgatggatgttgcctaatgag

SEQ ID NO: 41 - DNA, 24 bp, Artificial Sequence
  Description of Artificial Sequence Synthetic primer
ggaccctgactttactgctgtacc

SEQ ID NO: 42 - DNA, 21 bp, Artificial Sequence
  Description of Artificial Sequence Synthetic primer
ccgaaaccctgaggaggctcc

SEQ ID NO: 43 - DNA, 25 bp, Artificial Sequence
  Description of Artificial Sequence Synthetic primer
tctactgcaagaacggcggcttctt

SEQ ID NO: 44 - DNA, 25 bp, Artificial Sequence
  Description of Artificial Sequence Synthetic primer
cagtgccacataccaactggagtat

SEQ ID NO: 45 - DNA, 25 bp, Artificial Sequence
  Description of Artificial Sequence Synthetic primer
ccgaggagctttatgagatgctgag

SEQ ID NO: 46 - DNA, 25 bp, Artificial Sequence
  Description of Artificial Sequence Synthetic primer
agctgccactgtctcacacttgcat

SEQ ID NO: 47 - DNA, 20 bp, Artificial Sequence
  Description of Artificial Sequence Synthetic primer
cagtggagctggacaaagcc

SEQ ID NO: 48 - DNA, 20 bp, Artificial Sequence
  Description of Artificial Sequence Synthetic primer
tagcgacggttctggaacca

SEQ ID NO: 49 - DNA, 20 bp, Artificial Sequence
  Description of Artificial Sequence Synthetic primer
ctgtcatctcactatgggca

SEQ ID NO: 50 - DNA, 20 bp, Artificial Sequence
  Description of Artificial Sequence Synthetic primer
ccaagtccgagcaggaattt

SEQ ID NO: 51 - DNA, 20 bp, Artificial Sequence
  Description of Artificial Sequence Synthetic primer
agcaagaatacgatgccatc

SEQ ID NO: 52 - DNA, 20 bp, Artificial Sequence
  Description of Artificial Sequence Synthetic primer
gaaggggtggtggtacggtc

SEQ ID NO: 53 - DNA, 25 bp, Artificial Sequence
  Description of Artificial Sequence Synthetic primer
agtcggagatggcccagatacaaca

SEQ ID NO: 54 - DNA, 25 bp, Artificial Sequence
  Description of Artificial Sequence Synthetic primer
tccagcagttggatttcaagacggg

SEQ ID NO: 55 - DNA, 25 bp, Artificial Sequence
  Description of Artificial Sequence Synthetic primer
tacgtgctgaagatccagctgaagg

SEQ ID NO: 56 - DNA, 25 bp, Artificial Sequence
  Description of Artificial Sequence Synthetic primer
agttggaaggaccacatgcgtcgaa

SEQ ID NO: 57 - DNA, 24 bp, Artificial Sequence
  Description of Artificial Sequence Synthetic primer
ccaacacaaacaacagagggtgga

SEQ ID NO: 58 - DNA, 25 bp, Artificial Sequence
  Description of Artificial Sequence Synthetic primer
cgaccaggaacaatgacaccaagaa

SEQ ID NO: 59 - DNA, 25 bp, Artificial Sequence
  Description of Artificial Sequence Synthetic primer
caactactgcttcagctccacagag

SEQ ID NO: 60 - DNA, 25 bp, Artificial Sequence
  Description of Artificial Sequence Synthetic primer
aggagcgcacgatcatgttggacaa

SEQ ID NO: 61 - DNA, 25 bp, Artificial Sequence
  Description of Artificial Sequence Synthetic primer
tcggtgtgaacggatttggccgtat

SEQ ID NO: 62 - DNA, 25 bp, Artificial Sequence
  Description of Artificial Sequence Synthetic primer
agcccttccacgatgccaaagttgt

(19) United States
(12) Patent Application Publication
(10) Pub. No.: US 2007/0253937 A1
(43) Pub. Date: Nov. 1, 2007

(54) Novel multipotent stem cells and use thereof

(76) Inventors: Young-Sup Yoon, Watertown, MA (US); Douglas W. Losordo, Winchester, MA (US)

Correspondence Address: EDWARDS ANGELL PALMER & DODGE LLP, P.O. BOX 55874, BOSTON, MA, 02205, US

(21) Appl. No.: 11/412,416

(22) Filed: Apr. 26, 2006

(51) Int. Cl. A61K 35/14; C12N 5/08

(52) U.S. Cl. 424/93.7; 435/372

(57) ABSTRACT

Disclosed is an isolated bone marrow stem cell (BMSC) having undetectable or low levels of selected cell markers including those typical of endothelial, neuronal and smooth muscle cells. Also disclosed are grafts and pharmaceutical products that include such cells. Methods of making the BMSCs are also provided. The invention has a wide spectrum of useful applications including use in the prevention and treatment of cardiovascular disease.

### FIELD OF THE INVENTION

The invention generally relates to isolated bone marrow stem cells (BMSCS) having undetectable or low levels of selected cell markers. Importantly, the cells are multipotent and can be used to make a variety of desirable cell types. The invention has a wide spectrum of useful applications including use in the prevention and treatment of cardiovascular disease.

### BACKGROUND

There is increasing recognition that congestive heart failure (CHF) is a growing, worldwide epidemic. Several causes have been proposed, for instance, irreversible damage resulting from myocardial infarction (MI).

Infarct size is a major determinant of morbidity and mortality in CHF. For instance, large infarcts affect 40% or more of the left ventricle (LV) are typically associated with intractable cardiogenic shock or the rapid development of congestive heart failure. The longstanding axiom has been that the myocardium has a limited capacity for self repair or regeneration, and the irreversible loss of muscle and accompanying contraction and fibrosis of myocardial scar, sets into play a series of events, namely progressive ventricular remodeling of non-ischemic myocardium, that ultimately leads to progressive HF.

There have been efforts to understand HF in more detail. For instance, loss of cardiomyocyte (CMC) survival cues is thought to be associated HF. This suggests some importance in devising therapies to help maintain viable heart muscle cells during HF progression. Currently, no medication or procedure used clinically has shown efficacy in replacing myocardial scar with functioning contractile tissue. Therefore, given the major morbidity and mortality associated with MI and HF, new approaches have been sought to address the principal pathophysiologic deficits responsible for these conditions such as loss of vessels and CMCs.

There has been widespread belief that tissue specific stem cells could only differentiate into cells of the tissue of origin. However, there is emerging recognition that at least certain adult tissue-specific stem cells can differentiate into lineages other than the tissue of origin. Such plasticity has been referred to as trans-differentiation. Bone marrow cells appear to have the capacity to repopulate many non-hematopoietic tissues such as neuroectodermal cells, skeletal myoblasts, CMCs, endothelium, hepatic and biliary duct epithelium, and lung, gut and skin epithelia. Recently, a subset of BM derived stem cells referred to as multipotent adult progenitor cells (MAPCs) has been co purified with MSCs. MAPCs are thought to proliferate extensively and differentiate into cells of all three germ layers.

Therapeutic application of BM derived-stem cells has been attempted. Work has shown that endothelial progenitor cells (EPCs), angioblasts, or CD34(+) cells transplanted into ischemic myocardium incorporate into foci of neovascularization and have a favorable impact on LV function after MI. The more versatile potential of BM derived hematopoietic stem cells (HSCs) has been documented in other studies.

Another source of adult stem cells being explored for possible cardiac regeneration is non-hematopoietic mesenchymal stem cells (or marrow stromal cells, MSCs). MSCs can be derived from adult BM and have multi-lineage differentiation capacity. In culture, MSCs can maintain an undifferentiated, stable phenotype over many generations. In animal models of MI, it has also been documented that murine, bovine and human MSCs can undergo cardiomyogenic differentiation.

Although there have been reports that SCs derived from the BM have potential to regenerate myocardial tissues, it is unclear whether such immature cells have much clinical potential.

For example, while there is mounting evidence that BM contains populations of cells with plasticity, it is not certain that isolation and expansion of highly undifferentiated SCs from a single cell level will be successful. Clonal SC expansion is important as one way to enhance therapeutic reliability and efficacy. Moreover, past attempts to culture some BM cells such as MAPC has required use of expensive cytokine treatments making them less attractive for clinical use. More importantly, MAPC has shown minimal engraftment into the myocardium after being injected into the blastocyst. Certain BM cells such as Lin- c-KIT+ cells or angioblastshave been shown to regenerate a significant proportion of different myocardial tissues in vivo. However, these cells are quite rare and lack accepted culturing methods.

There have been other shortcomings associated with using BM particularly in therapeutic applications. For instance, it is not established whether a single human SC participates in both neovascularization and myogenesis in myocardial injury models. EC differentiation was not demonstrated with studies using human MSCs. Studies using MSCs demonstrated differentiation into cardiomyogenic phenotypes by immunohistochemical staining, however the number of incorporated cells were small and the morphologic features were not consistent with mature CMCs.

Accordingly, these and other drawbacks have limited use of BM as a source of cells that can be used to prevent, treat or reduce the severity of symptoms associated with heart disorders such as a myocardial infarction.

It would be desirable to have BM cells that are convenient to isolate and maintain in tissue culture as clonal isolates. It would be especially desirable if such BM cells could be maintained as pluripotent clones particularly as a source for at least one and preferably all three of EC, SMC and CMC cells. It would be even more desirable if such BM cells could be transplanted into diseased or damaged heart issue to help prevent, treat or reduce associated symptoms.

### SUMMARY OF THE INVENTION

We have discovered a new class of multipotent bone marrow stem cells (BMSCs) that can be cultured (expanded) from one or just a few marrow cells. Preferred BMSCs can proliferate for prolonged times and are capable of at least triple lineage differentiation. Formation of such cells from the BMSCs is controllable and can used to provide a potentially unlimited sources of differentiated cells and tissues including grafts. The invention has a wide spectrum of important applications including therapies involving transfer of the BMSCs (or cells derived therefrom) into a recipient in need of such treatment.

More specifically, we have isolated a new stem cell (SC) population from bone marrow (BM) that is multipotent (ie. plastic) and can proliferate in culture without detectable senescence or loss of plasticity for at least about 50 population doublings (PDs), more typically at least about 100 PDs, and usually about 140 PDs or more. In one aspect, we have clonally expanded BMSCs obtained from human bone marrow (referred to as hBMSCs) and found that they do not belong to any known BM-derived SC population such as hematopoietic SCs, mesenchymal SCs or multipotent adult progenitor cells such as endothelial progentior cells (EPCs). Importantly, preferred BMSCs of the invention are capable of differentiating into at least three different cell types when provided with suitable cues, particularly endothelial cells (ECs), smooth muscle cells (SMCs) and cardiomyocytes (CMCs). In this embodiment, the invention is well-suited to provide a potentially unlimited source of cells and tissue (including grafts) needed, for instance, to prevent, treat or reduce suffering associated with a range of cardiovascular disorders including those associated with infarcts.

Accordingly, and in one aspect, the invention provides an isolated population of bone marrow stem cells (BMSCs) that have undetectable or low (negligible) levels of at least one and preferably all of the following cell markers: CD90, CD117, CD34, CD113, FLK-1, tie-2, Oct 4, GATA-4, NKx2.5, Rex-1, CD105, CD117, CD133, MHC class I receptor and MHC class II receptor as determined by standard cell marker detection assay.

Such BMSCs have important uses and advantages. For instance, the BMSCs are highly plastic and can be used to provide a potentially unlimited source of cells and tissues to address cardiovascular disorders particularly those in which regenerative therapies are indicated. Although attempts have been made to use other SCs to address these disorders, such cells have been relatively less plastic then the BMSCs described herein. Patients have suffered for want of therapies that can provide a variety of cardiovascular cells and tissue. The invention addresses this need by providing, for the first time, BMSCs that can be readily converted into one or a variety of cardiovascular cells and particularly ECs, SMCs, and CMCs. In embodiments in which the BMSCs are isolated from a primate such as a human patient (hBMSCs) such cells can be maintained and/or treated ex vivo (eg., with one or more mitogens) and given back (transplanted) to the patient (or immunologically related individuals such as family members) to treat the cardiovascular disease. This feature of the invention helps reduce risk of unintended infection by viruses, for instance, and immunological rejection of the transplanted cells. Alternatively, or in addition, the BMSCs can be isolated from the patient and treated ex vivo (with or without mitogens) to produce tissue grafts that in particular embodiments are at least allogeneic and preferably syngeneic with respect to the patient. Such grafts can be used, for example, to address cardiovascular disorders in which relatively large amounts of the BMSCs or cells derived therefrom are needed.

Further provided by the invention is method of making the isolated bone marrow cells described herein (eg., hBMSCs). In one embodiment, the method includes at least one and preferably all of the following steps:
a) collecting bone marrow cells from a mammal which cells have a size of less than about 100 microns, preferably less than about 50 microns, more preferably about 40 microns or less,
b) culturing (expanding) the collected cells in medium under conditions that select for adherent cells,
c) selecting the adherent cells and expanding those cells in medium to semi-confluency,
d) serially diluting the cultured cells into chambers with conditioned medium, the dilution being sufficient to produce a density of less than about 1 cell per chamber to make clonal isolates of the expanded cells; and
e) culturing (expanding) each of the clonal isolates and selecting chambers having expanded cells to make the population of isolated bone marrow cells.

In another aspect, the invention provides tissue or a graft that includes the isolated BMSCs described herein such as, but not limited to, hBMSCs. Further provided is a cell culture, tissue or organ comprising the graft.

In yet another aspect of the invention, there is provided a method for preventing, treating or reducing the severity of a cardiovascular (heart) disorder. In one embodiment, the method includes administering to a mammal in need of such treatment at least one of the isolated BMSCs described herein (eg., hBMSCs). Preferably, the administration is sufficient to prevent, treat or reduce the severity of the disorder in the mammal. Alternatively, or in addition, the method includes administering to the mammal at least one graft of the invention under conditions that can help augment the heart disorder.

Further provided by the invention is a pharmaceutical product for preventing, treating or reducing the severity of a heart disorder. In one embodiment, the product includes at least one of the following components: a population of isolated BMSCs as described herein (eg., hBMSCs) and/or optionally directions for isolating the cells from a mammal; a graft of the invention and/or optionally directions for preparing, maintaining and/or using the graft; the cell culture, tissue or organ of the invention and/or optionally directions for preparing same from a mammal.

Also provided by the present invention is a population of isolated bone marrow cells obtained by at least one of and preferably all of the following process steps:
a) collecting bone marrow cells from a mammal which cells have a size of less than about 100 microns, preferably less than about 50 microns, more preferably about 40 microns or less,
b) culturing (expanding) the collected cells in medium under conditions that select for adherent cells,
c) selecting the adherent cells and expanding those cells in medium to semi-confluency,
d) serially diluting the cultured cells into chambers with conditioned medium, the dilution being sufficient to produce a density of less than about 1 cell per chamber to make clonal isolates of the expanded cells; and
e) culturing (expanding) each of the clonal isolates and selecting chambers having expanded cells to make the population of isolated bone marrow cells.

Other features, uses and advantages of the invention are described below.

### BRIEF DESCRIPTION OF THE DRAWINGS

FIG. 1A-E show characteristics of the Human Bone Marrow Stem Cells (hBMSC) of the invention.

FIG. 2A-D show in vitro differentiation of hBMSC into endothelial cell (EC) and smooth muscle cell (SMC) lineages.

FIGS. 3A-M show in vitro differentiation of hBMSC into neural and endodermal lineages.

FIGS. 4A-X show in vitro trans-differentiation and fusion of hBMSC to cardiomyocytes (CMC), EC and SMC cells.

FIGS. 5A-F show that hBMSC transplantation attenuates adverse cardiac remodeling in a rat model of myocardial infarction (MI).

FIG. 6A-T show engraftment and multilineage differentiation of transplanted hBMSC in infarcted myocardium.

FIG. 7A-U show that human BMSC transplantation augments myocardial cell proliferation and reduces myocardial apoptosis.

FIG. 8A-B shows that hBMSC transplantation upregulates mRNA expression of angiogenic cytokines and cardiac transcription.

FIG. 9A-L shows human BMSC transplantation increased capillary and CMC density and increased myocardial fibrosis.

FIG. 10 shows results of FACS analysis using multiple surface epitopes

### DETAILED DESRCRIPTION OF THE INVENTION

As discussed, the invention provides an isolated population of novel and multipotent BMSCs that have undetectable or low (negligible) levels of at least one and preferably all of the following cell markers: CD90, CD117, CD34, CD113, FLK-1, tie-2, Oct 4, GATA-4, NKx2.5, Rex-1, CD105, CD117, CD133, MHC class I receptor and MHC class II receptor. Such markers can be readily determined by what is referred to herein as a standard cell marker detection assay. The BMSCs of the invention have a wide spectrum of important uses including use in the prevention, treatment or alleviation of symptoms associated with a cardiovascular disorders, particularly those coronary diseases directly or indirectly associated with ischemia (myocardial ischemia), an infarct (myocardial infarction), congestive heart failure (CHF) and related coronary indications.

By the phrase “standard cell marker detection assay” is meant a conventional immunological or molecular assay formatted to detect and optionally quantitate one of the foregoing cell markers (ie., CD90, CD117, CD34 etc.). Examples of such conventional immunological assays include Western blotting, ELISA, and RIA. Preferred antibodies for use in such assays are provided below. See generally, Harlow and Lane in Antibodies: A Laboratory Manual, CSH Publications, N.Y. (1988), for disclosure relating to these and other suitable assays. Particular molecular assays suitable for such use include polymerase chain reaction (PCR) type assays using oligonucleotide primers disclosed herein (see Table 1, for instance). See WO 92/07075 for general disclosure relating to recombinant PCR and related methods. See also Sambrook et al. in Molecular Cloning: A Laboratory Manual (2d ed. 1989); and Ausubel et al., Current Protocols in Molecular Biology, John Wiley & Sons, New York, 1989; for general disclosure relating to recognized immunological and molecular assays that can be used to detect the cell markers.

By “isolated” as it is used to refer to BMSCs is meant that the cells have been separated from bone marrow and other cell substituents that naturally accompany it. Preferably, the BMSCs of the invention are at least 80% or 90% to 95% pure (w/w). BMSC and particularly hBMSC having at least 98 to 99% homogeneity (w/w) are most preferred for many pharmaceutical, clinical and research applications. Once substantially purified or isolated, the BMSC would be substantially free of unwanted marrow contaminants. Once purified partially or to substantial purity, the BMSC are suited for therapeutic or other uses such as those provided herein. Purity can be determined by a variety of standard techniques such as cell culture, microscopical and centrifugation techniques (eg., Ficoll gradient).

Particular disclosure relating to isolating and manipulating endothelial cells (ECs) and especially endothelial progenitor cells (EPCs) can be found eg., in U.S. Pat. No. 5,980,887 and PCT/US99/05130 (WO 99/45775).

By the term “mammal” is meant a primate, domesticated or other mammal such as a rodent or rabbit. A preferred primate is a chimpanzee, monkey or a human patient in need of treatment. Suitable domesticated animals include gerbils, horses, dogs, cats, goats, sheep, pigs, chickens and the like. A preferred rodent is a rat or mouse. A preferred BMSC is isolated from a primate and particularly a human subject such as those in need of cardiovascular therapy.

More particular BMSC in accord with the invention are essentially spherical as determined by inspection (typically microscopy) and have a diameter of less than about 25 to 35 micrometers, preferably less than about 15 micrometers. Other particular cells of the invention will have a mean telomere restriction fragment length (TRF) of less than about 30 to 40 kilobases, preferably less than about 20 kilobases such as about 17 kilobases. Preferred methods for measuring the diameter of the BMSC and determining TRF are provided below.

Other particular BMSC of the invention are essentially euploid which euploidy is essentially maintained for at least about 10 passages in cell culture, preferably between from about 20 to about 200 passages. Methods for determining cell ploidy are known and are provided below.

More specific BMSCs in accord with the invention are capable of forming endothelial cells (ECs), for instance, after contact with EC promoting conditions as determined by a standard EC differentiation assay. Examples of such EC promoting conditions are known in the field and include contact with certain angiogenic factors and cell mitogens such as those disclosed by the U.S. Pat. No. 5,980,887; PCT/US99/05130 (WO 99/45775) and references cited therein. Such disclosed factors and mitogens include acidic and basic fibroblast growth factors (aFGF and bFGF), vascular endothelial growth factor (VEGF-1), VEGF165, epidermal growth factor (EGF), transforming growth factor α and β (TGF-α and TFG-β), platelet-derived endothelial growth factor (PD-ECGF), platelet-derived growth factor (PDGF), tumor necrosis factor α (TNF-α), hepatocyte growth factor (HGF), insulin like growth factor (IGF), erythropoietin, colony stimulating factor (CSF), macrophage-CSF (M-CSF), granulocyte/macrophage CSF (GM-CSF), angiopoetin-1 (Ang1) and nitric oxidesynthase (NOS); and functional fragments thereof. See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994). Muteins or fragments of a mitogen may be used as long as they induce or promote formation of ECs.

A preferred EC promoting condition includes contact with VEGF, particularly VEGF-1, VEGF165, or both. Additionally preferred EC promoting conditions include contact with certain cell matrix proteins such as fibronectin. See Example 2 below.

By the phrase “standard EC differentiation assay” is meant any of the assays used to detect and monitor function of ECs such as those disclosed by the U.S. Pat. No. 5,980,887 and WO 99/45775. A preferred assay involves detection of EC specific markers such as reported in Example 2.

Also preferred are BMSCs that can form smooth muscle cells (SMCs), particularly after contact with SMC promoting conditions as determined by a standard SMC differentiation assay. Typical SMC promoting conditions are known and include contact with at least one angiogenic factor or cell mitogen as discussed previously. A preferred SMC promoting condition involves contact with a platelet derived growth factor (PDGF) including muteins or active fragments thereof.

By the phrase “standard SMC differentiation assay” is meant an immunological or molecular test (eg., ELISA, Western blot or PCR) that is capable of detecting and optionally quantitating at least one and preferably all of the following SMC specific markers: αSMA, PDGFβ receptor, SM22α, and SMI. An illustration of an acceptable assay is provided by Example 2.

Still further BMSCs in accord with the invention can form neuronal cells especially after contact with neuronal cell promoting conditions as determined by a standard neuronal cell differentiation assay. A variety of neuronal cell promoting conditions are known and include contact with one or more of the forgoing factors and mitogens. Preferred are conditions that involve contact with hepatocyte growth factor (HGF) alone or preferably in combination with fibroblast growth factor 4 (FGF-4). Still further preferred neuronal cell promoting conditions involve further contact with DMSO or a pharmaceutically acceptable butyrate salt such as sodium or potassium salts thereof. Still further preferred neuronal cell promoting conditions include contact with a suitable cell matrix protein such as poly-L-omithine-laminin.

By the phrase “standard neuronal cell differentiation” assay is meant an immunological or molecular test (eg., ELISA, Western blot or PCR) that is capable of detecting and optionally quantitating at least one and preferably all of the following neuronal specific markers: GFAP, GalC, NF200, a tubulin, myelin basic protein, MAP2, GAD and Tau. See Example 2 (disclosing a particularly preferred neuronal cell promoting condition).

Additionally suitable BMSCs of the invention include those cells that can form cardiomyocytes after contact with accessory cardiomyocytes. By the term “accessory cardiomyocytes” are meant cells that were cardiomyocytes prior to the contact with the BMSCs. Examples of such accessory cardiomyocytes include established cultures of such cells as well as cardiomyocytes inhabiting cardiac tissue. In some embodiments, the formation of cardiomyocytes by the BMSCs will be assisted by cell fusion between the stem cells and the accessory cardiomyocytes. Such cardiomyocytes (and particularly the accessory cells) can be maintained by one or a combination of stratagies including those involving maintenance in vitro, ex vivo or in vivo.

As discussed, it is an object of the invention to provide a graft that includes (or in some embodiments consists of) the isolated bone marrow stem cells described herein. By “graft” is meant a cell or tissue preparation that includes the BMSCs and optionally other cells such as ECs, SMCs and CMCs from a mammal. A donor is defined as the source of the BMSCs whereas the recipient is the subject that receives the graft. Immunological relationship between the donor and recipient can be allogenic, autologous, or xenogeneic as needed. In preferred invention embodiments, the donor and recipient will be genetically identical and usually will be the same individual (syngeneic). In this instance, the graft will be syngeneic with respect to the donor and recipient.

By “graft” is also meant BMSCs of the invention that have been administered to a recipient and become part of one or more tissues or organs of that recipient. Sometimes the word “engraftment” will be used to denote intended assimilation of the BMSCs into a targeted tissue or organ (either as BMSCs or differentiated cells). Preferred engraftment involves a cardiovascular tissue such as veins, arteries and particularly cardiac tissue. A graft of the invention may also take the form of a tissue culture preparation in which BMSCs of the invention have been combined with other cells and or mitogens to promote differentiation and/or cell replication that produces an intended graft. If desired, the preparation can be combined with synthetic or semi synthetic fibers to give structure to the graft. Fibers such as Dacron, Teflon or Gore-Tex are preferred for certain applications.

A particular example of a graft of the invention is a preparation of hBMSCs that have been isolated from a donor to prevent, treat or reduce the severity of a cardiovascular disorder such as myocardial ischemia or an infarct. The preparation can include a pharmaceutically acceptable carrier such as saline and optionally may include at least one of mitogens, angiogenic factors, CMCs, ECs, EPCs, and SMCs to assist an intended engraftment result.

Particular CMCs of the invention express CMC specific protein (cTnI). Preferred are ECs that ILB-4. Of specific interest are SMCs that express α-SMA and related markers.

As discussed, the invention further provides a method for making the BMSCs described herein including those cells obtained from human patients (hBMSCs). In one embodiment, the method includes at least one and preferably all of the following steps:
a) collecting bone marrow cells from a mammal (preferably a young adult) which cells have a size of less than about 100 microns, preferably less than about 50 microns, more preferably about 40 microns or less,
b) culturing (expanding) the collected cells in medium under conditions that select for adherent cells,
c) selecting the adherent cells and expanding those cells in medium to semi-confluency,
d) serially diluting the cultured cells into chambers with conditioned medium, the dilution being sufficient to produce a density of less than about 1 cell per chamber to make clonal isolates of the expanded cells; and
e) culturing (expanding) each of the clonal isolates and selecting chambers having expanded cells to make the population of isolated bone marrow cells.

More specifically, and in embodiments in which human cells are desired, the hBMSC can be obtained by taking fresh unprocessed bone marrow (BM) cells from young male donors. Alternatively, such cells can be purchased. The cells are typically separated from blood cells by centrifugation, hemolysis and related standard procedures. The BMs are washed in an acceptable buffer such as DPBS and filtered to collect cells having a size less that about 100 microns, preferably less than about 50 microns, more preferably about 40 microns. A standard nylon filter, for instance, can be used. Once isolated, the BMs are grown on a complete culture medium with low glucose (eg., DMEM) that contains a rich source of growth factors and cytokines. Fetal bovine serum (FBS) is preferred. Cells are cultured (ie. expanded) for less than about two weeks, preferably about a week or less such as four to six days. The conditioned medium is then replaced with fresh medium, adherent cells are removed from the culture dishes and resuspended in fresh medium to select cells that can be expanded. The selected cells are grown to semiconfluency (between 50% to 90% confluent) and again, adherent cells are selected. Such cells are then reseeded in complete medium in a tissue culture flask at a density of about 10⁴ cells per centimeter. After the cells reach semiconfluency, they are reseeded (serially) into the flasks at the same or similar density. The cultures are preferably passaged more than one time, typically less than five times and preferably about two times to continue selection for expanding cells. Selected cells are then serially diluted into single well chambers (eg., standard 96 well plate) at a density of less than about 1 cell per chamber, preferably ½ a cell per chamber. Preferably, the cells are cultured with conditioned media to promote growth to sub confluence (ie. less then 50% confluent). Wells with expanded cell clones were expanded and replated as needed.

In a particular embodiment, the method further includes selected cell clones that do express detectable levels of at least one of the following markers: CD90, CD117, CD34, CD113, FLK-1, tie-2, Oct 4, GATA-4, NKx2.5, Rex-1, CD105, CD117, CD133, MHC class I receptor, MHC class II receptor or other cell marker as described herein. Methods for performing the selection include any of the suitable assays disclosed herein. In embodiments in which larger amounts of stem cells are needed a more automated or semi-automated method will often be preferred such as fluorescence activated cell sorting (FACS). See the Examples provided below.

A preferred example of the method for making the BMSCs is provided by Example 1 below.

The invention also provides a method for preventing, treating or reducing the severity of a heart disorder that in one embodiment includes administering to a mammal in need of treatment at least one of the isolated BMSCs, grafts, or both disclosed herein. Preferably, the administration is sufficient to prevent, treat or reduce the severity of the disorder in the mammal.

In one embodiment, the method further includes incubating the cells or graft in the mammal for at least about a week, preferably between from about two to eight weeks. It will be apparent to those working in the field that the incubation period is flexible and can be extended or shorten to address a particular indication or with respect to the health or age of the individual in need of treatment. Typical amounts of BMSC to use will depend on these and other recognized parameters including the disease to be treated and the speed of recovery needed. However for most applications between from about 10³ to about 10⁷ BMSCs will suffice, typically about 10⁵ of such cells. Cells may be administered by any acceptable route including suspending the cells in saline and administering same with a needle, stent, catheter or like device. In embodiments in which myocardial ischemia or an infarct is to be addressed, the administration will be a bolus injection near or directly into the site of injury.

A particular method for regenerating heart tissue following myocardial ischemia is provided by Examples 3 and 4. See also Examples-5-6 (showing engraftment of hBMSC into cardiac tissue). As discussed, transplantation of the BMSCs provides a variety of therapeutic effects including boosting levels of angiogenic factors and increasing capillary and CMC densities.

In another embodiment, the method further includes administering to the mammal in need of treatment at least one angiogenic factor or mitogen (or functional fragment of the factor or mitogen). Preferred angiogenic factors and mitogens (and methods of use) are disclosed herein as well as U.S. Pat. No. 5,980,887 and WO 99/45775. Alternatively, or in addition, the method can include administering to the mammal at least one nucleic acid encoding at least one angiogenic factor or functional fragment thereof. Method for administering such nucleic acids to the mammal have been disclosed by U.S. Pat. No. 5,980,887 and WO 99/45775, for instance. Treatment with the angiogenic factor/mitogen protein or nucleic acid encoding same can precede use of the BMSCs or it can be used during or after such treatment as needed.

In yet another embodiment, the method further includes administering to the mammal endothelial progenitor cells (EPCs). This invention embodiment especially finds use where good vascular growth is needed to address a cardiovascular disorder. Methods for making and using EPCs have been disclosed. See U.S. Pat. No. 5,980,887, for example. Typical methods can include isolating the EPCs from the mammal and contacting the EPCs with at least one angiogenic factor and/or mitogen ex vivo.

As discussed, the invention is applicable to the prevention and treatment of a wide variety of cardiovascular disorders including congestive heart failure (CHF), ischemic cardiomyopathy, myocardial ischemia, and an infarct. If desired, such methods can further include monitoring cardiac function in the mammal seeking treatment, eg., by monitoring at least one of echocardiography, ventricular end-diastolic dimension (LVEDD), end-systolic dimension (LVESD), fractional shortening (FS), wall motion score index (WMSI) and LV systolic pressure (LVSP). Preferred invention methods involving prevention or treatment of a particular cardiovascular disease will manifest good cardiac function as exemplified by one or more of these tests. By “good” is meant at least a 10% improvement, preferably at least 20% or 30% compared to a control that does not receive an invention composition (or receives a placebo). Particular methods for performing these tests are known and are described in the Examples section.

Further provided by the invention is a pharmaceutical product for preventing, treating or reducing the severity of a heart disorder that includes, for instance, at least one of the following components: BMSCs, preferably hBMSCs and optionally directions for isolating the cells from a mammal; a graft as disclosed herein, preferably one with hBMSCs or cells or tissue derived from same and optionally directions for preparing, maintaining and/or using the graft; the cell culture, tissue or organ and optionally directions for preparing same. In one embodiment, the product further includes at least one angiogenic factor, mitogen; or functional fragment thereof. In another embodiment, the product further comprises at least one nucleic acid encoding the angiogenic factor, mitogen or functional fragment thereof.

As also discussed, the invention provides isolated BMSCs such as hBMSCs that are made by a method disclosed herein. Such cells possess advantages such as desirable plasticity and ability to propagate for long periods of time without significant culture problems (eg., undesired polyploidy and loss of mulipotency). In one embodiment, such cells are further made by collecting cells that do not express detectable levels of at least one of the following markers: CD90, CD117, CD34, CD113, FLK-1, tie-2, Oct 4, GATA-4, NKx2.5, Rex-1, CD105, CD117, CD133, MHC class I receptor, MHC class II receptor or other marker as provided herein.

The present disclosure particularly shows that the invention can be used to isolate, from a single cell, BMSCs and especially hBMSCs that possess potentially unlimited proliferation capacity and are capable of triple lineage differentiation including ECs, SMCs and CMCs. The transplantation of hBMSC into acutely infarcted myocardium attenuates cardiac dysfunction both by de novo differentiation into myocardial tissues and favorable paracrine effects on the adjacent cells.

More specifically, the present disclosure including the Examples show that a novel stem cell (SC) population (pop) has been identified within adult human bone marrow (BM). Further shown is isolation of these cells at the single cell level and proliferation without obvious senescence or loss of multi-potency for >140 population doublings (PDs). These clonally expanded human BM-derived multipotent SC (hBMSC) have minimal to no detectable expression of CD90 and CD117. Thus, the hBMSC that we have isolated do not belong to any known BM-derived SC pop such as hematopoietic SCs, mesenchymal SCs or multipotent adult progenitor cells. hBMSC exhibit a potential for differentiation (Diff) into cells of all three germ layers in vitro. Co-culture of hBMSC with neonatal rat cardiomyocytes (CMC), rat aortic endothelial cells (EC) or rat smooth muscle cells (SMC) revealed phenotypic changes consisting of both true Diff and cell to cell fusion. We tested whether acutely infarcted myocardium (Myoc) could be restored by transplantation (Xplnt) of hBMSC. Immediately after inducing MI in nude rats, hBMSC were xplnt'd in the wall of peri-infarct area. Myoc function measured by echo and pressure transducer was significantly better in the hBMSC xplnt rats than in the total BM cell xplnt or saline injected control rats 28 days later. Robust engraftment of xplnt'd cells was observed, which exhibited Diff into CMCs, ECs and SMCs. In the hBMSC transplanted Myoc, multiple angiogenic cytokines and essential cardiac transcription factors were significantly upregulated and higher rates of EC and CMC proliferation were observed. Conclusion: a novel multipotent SC population exists within the human BM, and locally xplnt'd hBMSC can ameliorate the outcome of acute MI by de novo vasculogenesis and myogenesis as well as augmenting proliferation and preservation of host myocardial tissues. This is the first demonstration that human BM-derived SCs can differentiate into all the elements required for the regeneration of ischemic myocardium both in vitro and in vivo.

As discussed, the disclosure shows that a novel adult human stem cell population, hBMSC, which does not belong to the known population of adult stem cells, was isolated beginning at the single cell level from mixed total BM cell culture and expanded clonally in culture for >140 PDs without loss of plasticity or onset of replicative senescence. Further, clonally derived hBMSC differentiated into cells of three germ layers (endoderm, mesoderm, neuroectoderm) in vitro. Also, the transplantation of hBMSC into an animal model of MI ameliorates the functional and pathologic changes following MI. Additionally, the mechanism of improved cardiac function consists not only of differentiation into essential myocardial tissues such as CMCs, ECs and SMCs, but also involves apparent paracrine effects of the transplanted stem cells, which stimulate the proliferation of host myocardial tissues and prevent apoptosis of endangered cells following ischemic injury.

The hBMSC that we have identified are a unique population of adult BM derived multipotent SCs in that <1% of hBMSC expresses CD90 and CD117. All the common marker panels defining the HSC, MSC, and MAPC do not match the profile of hBMSC. hBMSC do not express the well known MSC marker proteins, CD105(SH2) and CD73(SH3 and SH4) even though the media used is similar to those of MSC cultures {Barry, 1999; Barry, 2001}. The absence of or minimal expression of surface molecules is a pre-requisite for the plasticity of hBMSC as shown in other adult multipotent stem cells[Jiang, 2002]{Colter, 2001}. The unique surface phenotype of hBMSC results from the use of total BM cell population for initial culture and the clonal isolation/selection procedure. Our invention has addressed one important issue in stem cell biology, i.e. whether multipotent stem cells could be culture-expanded from a single cell. Here, we first demonstrate that in vitro and in vivo differentiation of hBMSC to mesoderm, endoderm and neuroectoderm occurs from clones developed from a single cell. In contrast to MAPCs, hBMSC do not express genetic markers of ESCs, such as Oct-4 and Rex-1, which are believed to be the factors essential for MAPCs. Our work suggests that the factors involving the maintenance of the undifferentiated state and unrestricted proliferative potential of adult SCs differ from those of ESCs. Another difference is the fact that rat BMSCs did not require leukemia inhibitory peptide for culture expansion{Yoon YS, 2002}, which is essential for MAPC cultures. Rather, maintenance of the low cell density plays an important role in maintaining multi-potency and expandability of hBMSC.

Recent studies demonstrated an important role of cell fusion for stem cell plasticity. The present in vitro data indicate that both fusion and trans-differentiation are responsible for the phenotypic changes of hBMSC to ECs, SMCs and CMCs and that the prevalence depends on cell type. Although no definitive studies were performed in vivo to quantify the contribution of cell fusion to phenotypic changes observed, the results of in vitro studies implied that both fusion and transdifferention are likely to play a role. Whatever the contribution from each component, transplanted hBMSC helped to reconstitute myocardial integrity after ischemic injury, just as the fused hepatocytes did for metabolic liver disease. It is infered from this work that cell fusion is not necessarily a critical barrier for therapeutic application, at least for adult SCs.

Compared to the other SCs, the hBMSC of the invention provide advantages for regenerative therapy of cardiac diseases. Among the stem cells shown to improve cardiac function are EPCs, CD34(+) cells, angioblasts, that have only shown to promote neovascularization in vivo. Multipotent stem cells, such as MSCs, c-kit positive lineage negative cells, SP cells, and ESC have been tested in ischemic animal models. ESCs have only demonstrated the potential to differentiate into CMCs but have not been shown to differentiate into ECs or SMCs in animal models of IHD. Moreover, the use of ESCs is hindered by certain ethical issues which remain to be resolved prior to clinical application. The trans-differentiation of SP cells into multiple lineages in IHD was demonstrated only with mouse cells but not with human cells, and the therapeutic effect is unknown. Also, mouse but not human Lin(−) c-kit(+) cells have shown multi-potency and therapeutic effect but the scarcity of these SC in the circulation or BM and the unavailability of culture-expansion limits their therapeutic use. Human MSCs have been shown to transdifferentiate into CMCs but the morphologic appearance differs from those of organized CMCs, and EC and SMC differentiation was not demonstrated. In contrast, the hBMSC described herein have the required multi-potency in vitro and in vivo to regenerate damaged myocardium, are culture-expandable and disclose the functional capability for therapeutic application.

Coronary heart disease accounts for 50% of all cardiovascular deaths and nearly 40% of the incidence of heart failure. The current findings have provided compelling evidence that the invention can be used to prevent, treat or reduce the severity of a variety of heart disorders including those associated with infarcts and/or ischemia. More specifically, the invention promotes de novo vasculo-myogenesis and augments angiogenesis and proliferation of existent CMCs, reduces progressive fibrosis, and improves ventricular function in a rodent model of MI. We believe this is the first demonstration that adult human stem cells can promote successful treatment of acute MIs during the process of ischemic tissue damage. The invention is also applicable to the prevention or treatment of IHD, particularly to improve the immediate and long-term outcome of IHD.

The following Examples are intended to be illustrative and not limit the invention. All references disclosed herein are incorporated by reference.

### EXAMPLE 1

### Culture and Characteristics of hBMSC

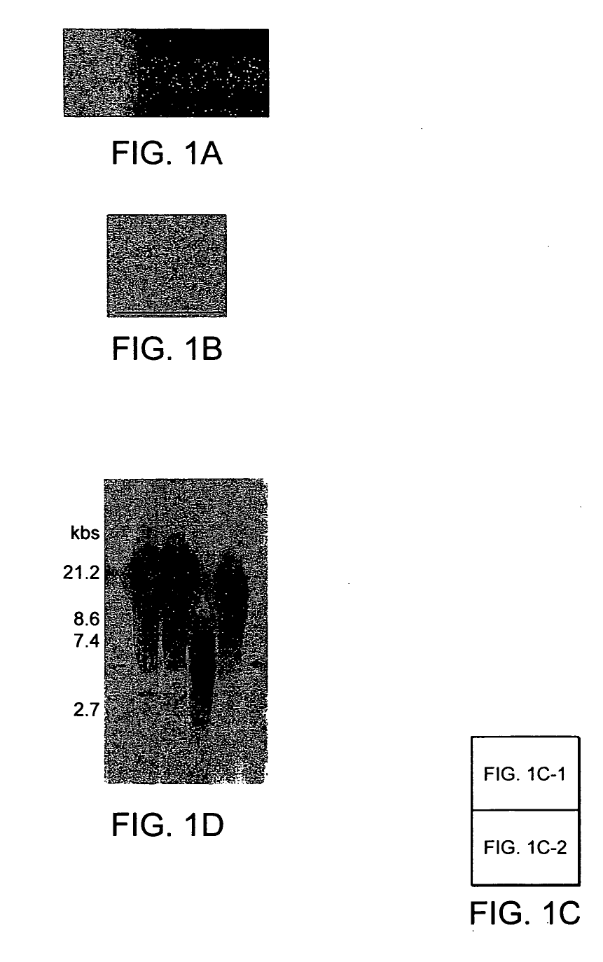

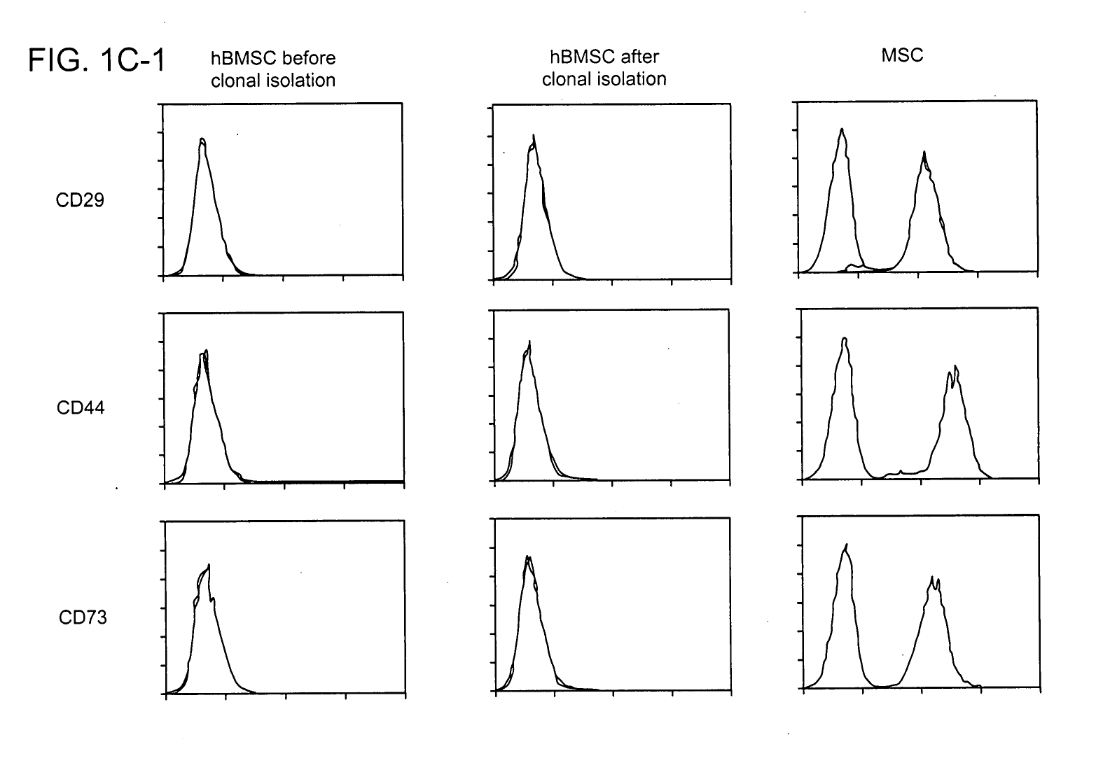

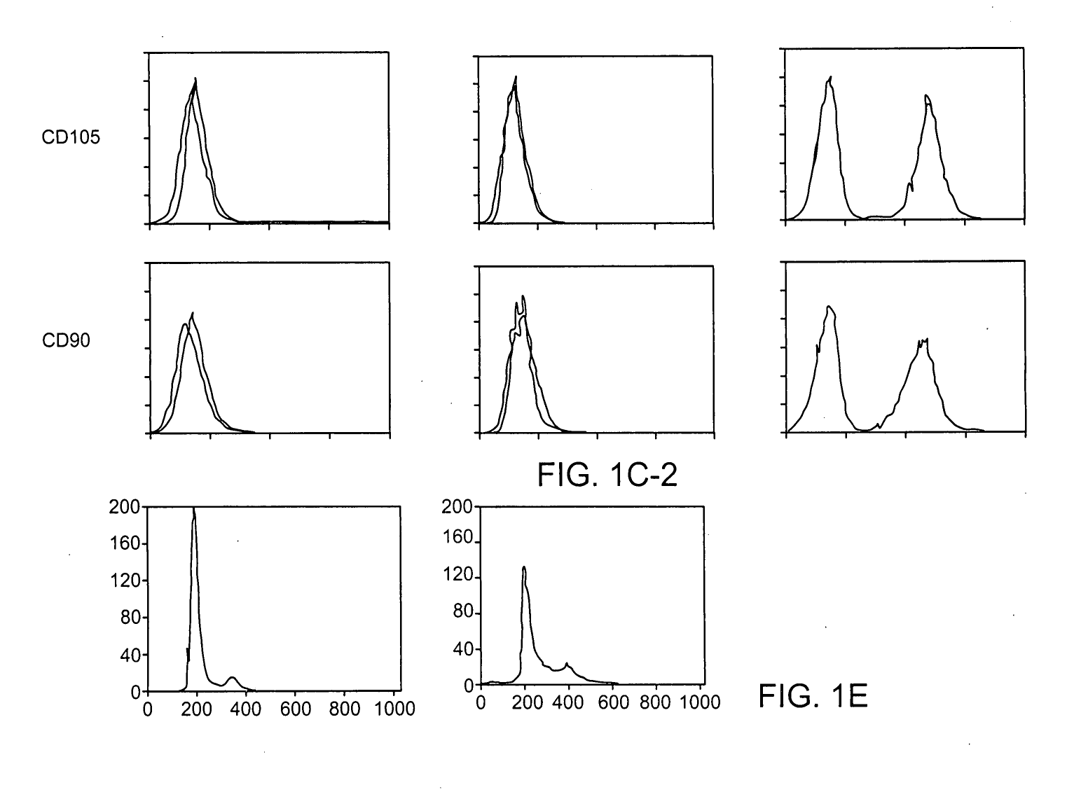

The clonality, surface epitopes, euploidy and proliferation of hBMSCs was evaluated as follows. Fresh unprocessed human BMs from young male donors were purchased. Three different marrow specimens from three different donors for SC cultures were used. After serial culture of total marrow cells in plastic dishes in Dulbecco's modified eagle's medium (DMEM) with low (1 g) glucose containing 17% of fetal bovine serum (FBS), cells were labeled with red fluorescent dye, DiI. After limiting dilution (1-2 cells per well in 96 well plate) wells containing a single cell visualized by fluorescent microscopy were selected. Of wells containing a single cell (FIG. 1a), 6±4% (range 2-13%) demonstrated survival and proliferation of cells. When cells were grown to 40-50% confluence, cells from each well (one clone) were reseeded into one well of 6-well plates and thereafter serially reseeded in 25 cm² tissue culture flask (T25), T75 and T175 at a density of 4-8×10³ cells/cm², respectively. Subsequently, cells were cultured at a density of 4-8×10³ cells/cm² in T175 and replated 1:20-40 dilution. Reseeding was performed in triplicate and the most rapidly growing clones were selected in every culture and expanded in serial cultures. After obtaining more than 10 clones from each bone marrow at passage 6, two clones were selected for continuous cultures. Morphologically, compared to the MSCs, hBMSC are more spherical cells, are smaller in size (<15 μm in diameter) and exhibit a high nucleus to cytoplasm ratio (FIG. 1b). Clonal cell lines derived in this manner have undergone more than 140 population doublings (PDs). The doubling time was 38±9 hrs between 20 to 80 PDs. FACS analysis using multiple surface epitopes demonstrated minimal to no expression (0-1%) of CD90 (FIG. 1c) and CD117 (FIG. 1S). hBMSC did not differentiate spontaneously and maintained their phenotype during culture expansion. In contrast, prior to clonal isolation the cultured hBMSC expressed low levels of CD105, CD90 and CD117 while mesenchymal stem cells expressed high levels of CD29, CD44, CD73, CD105 and CD90 (FIG. 1b). Major histocompatibility complex (MHC) class I(ABC) and II(DR) molecules and known hematopoietic stem markers (CD34, CD133, FLK-1, Tie2) were not expressed (FIG. 1S). RT-PCR analysis was negative for Oct4 and Rex-1, which are known markers of embryonic stem cells and MAPCs. These results indicated that hBMSC do not belong to the known HSCs, MSCs or MAPCs and are immunologically inert. Mean telomere restriction fragment (TRF) length of hBMSC cultured for 5 PDs was about 17 kilobases (kb); when re-tested after 120 PDs, mean TRF remained unchanged (FIG. 1d). DNA ploidy (the number of DNA copies) was examined by FACS analysis after staining DNA with propidium iodide. hBMSC cultured for 20 and 140 PDs from 3 different clones demonstrated no evidence for increased ploidy, suggesting that the euploidy is maintained during the culture-expansion.

FIG. 1A-E are discussed in more detail as follows: a. Phase contrast and fluorescent images show a single cell per well. b. Morphologically, many hBMSC show round morphology with a cell size of <15 μm in diameter. Scale bar=50 μm c. Clonally isolated hBMSC cultured for 140 PDs were labeled with PE or FITC-conjugated antibodies against human CD29, CD44, CD73, CD105, CD90 or immunoglobulin isotype controls. Cells were analyzed with a FACStar flow cytometer (B-D). Blue line, control immunoglobulin; red line, specific Ab. The cultured hBMSC prior to clonal isolation expressed low levels of CD105, CD90. Clonally isolated hBMSC only showed none to minimal expression (0-1%) of CD90 (FIG. 1c). In contrast, the purchased mesenchymal stem cells expressed high level of CD29, CD44, CD73, CD105 and CD90. d. Mean telomere restriction fragment (TRF) length of hBMSC cultured for 10 PDs(lane 1, kb) and 120 PDs(lane 2, kb) showing no difference in mean TRF. e. DNA ploidy analysis. hBMSC before and after clonal selection were stained with propidium iodide and subjected to FACS analysis. hBMSC cultured for 20 (left panel) and 140 (right panel) PDs demonstrated <1% of over-diploid DNA content. A representative example of >3 experiments.

### EXAMPLE 2

### Plasticity of hBMSC: In Vitro Differentiation

The in vitro differentiation potential of hBMSC was tested by adopting and modifying previously published culture conditions for adult and embryonic stem cells, which primarily requires lineage-specific cytokines.

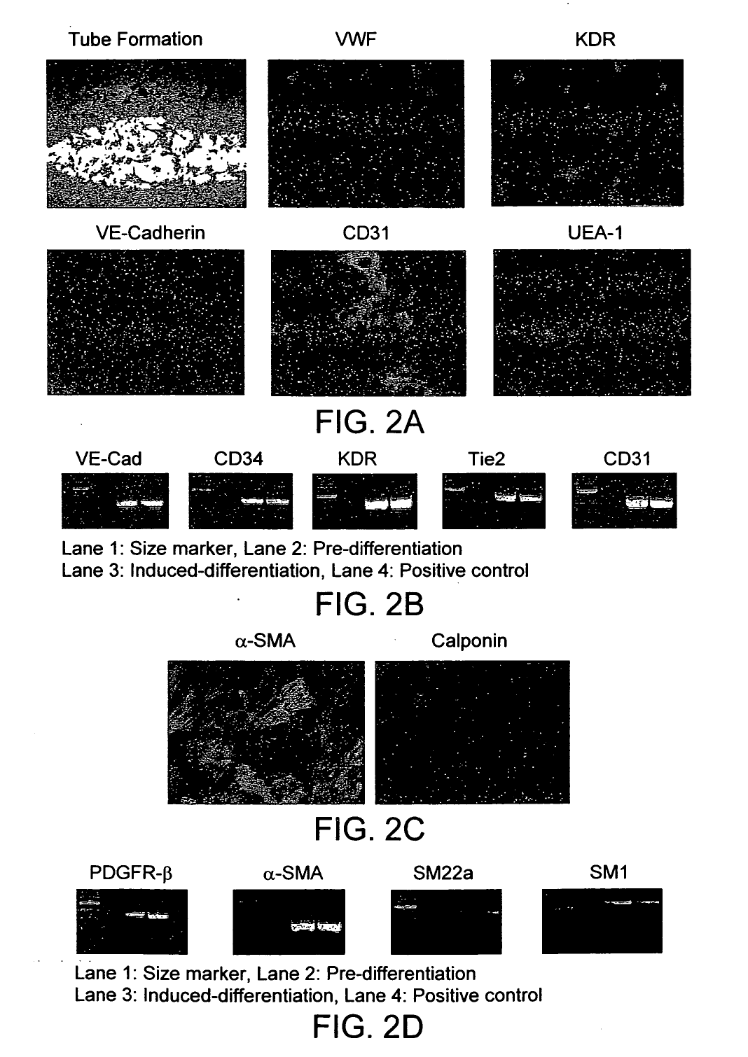

To induce differentiation into ECs, hBMSC were replated at 5×10⁴ cells/cm² in either 0.1% gelatin or fibronectin coated glass chamber slides in DMEM or EBM-2 (Clonetics) with 2% FBS, 10⁻⁸ dexamethasone and 10 ng/ml VEGF. Five days after culture, hBMSC formed vascular tube-like structures (FIG. 2a, left upper panel). Fourteen days after culture, hBMSC exhibited EC specific phenotypes such as von Willebrand factor (vWF), Flk1, VE-Cadherin, CD31, human-specific EC marker Ulex europaeus lectin type 1 (UAE-1) (FIG. 2a). At 14 days, 63±8% (VE-Cadherin) to 85±12% (UAE-1) of hBMSC demonstrated EC phenotypes by immunocytochemistry. RT-PCR of EC specific genes, VE-Cadherin, CD34, Flk-1, Tie2 and CD31 also confirmed the differentiation of hBMSC (pre-differentiation) into EC phenotypes (post-differentiation) (FIG. 2b).

To induce differentiation into SMC lineages, hBMSC were replated at 1×10⁵ cells/cm² in non-coated or fibronectin coated plastic dishes in 1-2% DMEM or EBM-2 supplemented with PDGF-BB(50 ng/ml, R&D) [Hellstrom, 1999; Yamashita, 2000]. Fourteen days after culture, 89±6% and 67±12% of hBMSC stained positive for α-smooth muscle actin (α-SMA) and calponin, respectively, indicative of differentiation into SMC phenotypes (FIG. 2c). RT-PCR confirmed the de novo expression of SMC specific genes, αSMA, PDGFβ receptor, SM22α, and SM1 (FIG. 2d).

FIG. 2A-D are explained in more detail as follows: a. Hoffman image (left upper) 5 days after culture with DMEM in gelatin coated glass chambers that, hBMSC formed typical vascular tube-like structures. Immunfluorscent imaging demonstrates hBMSC exhibits EC specific proteins, such as vWF, Flk1, VE-Cadherin, CD31 and UEA-1 after culturing hBMSC in EC differentiation media for 14 days. b. RT-PCR analysis using EC specific gene primers, VE-Cadherin, CD34, Flk-1, Tie2 and CD31 also confirmed the differentiation of hBMSC (pre-differentiation) into EC phenotypes (post-differentiation) c. hBMSC cultured in 2% DMEM containing PDGF-BB for 14 days demonstrates expression of SMC specific proteins, α-SMA and calponin by IF staining. d. RT-PCR analysis shows that SMC specific genes, PDGFR-β, α-SMA, SM22α and SM1 are only expressed after induction of differentiation. Thick ladder in size marker in RT-PCR (b and d)=600bp

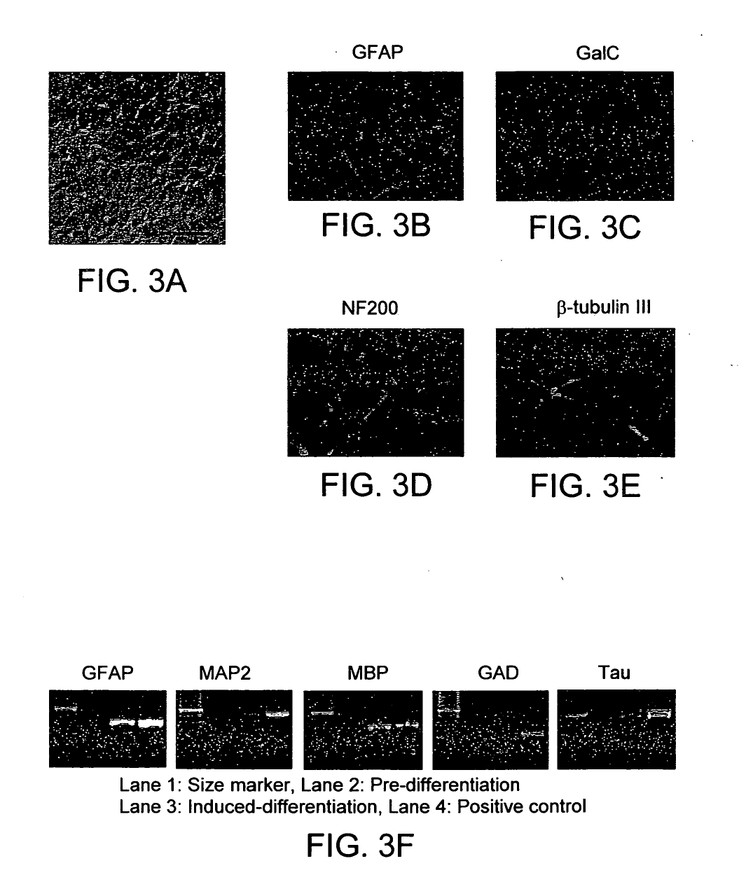

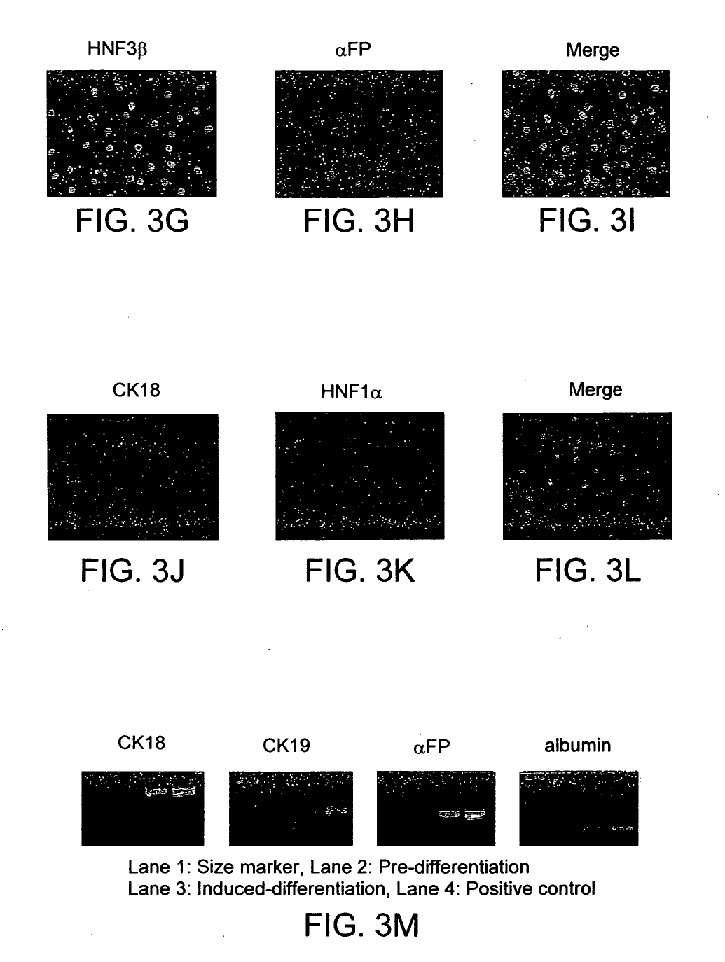

To induce neural lineage differentiation, hBMSC were plated at 4×10⁴/cm² on plastic dishes or Poly-L-omithine-laminin coated dishes with 100 ng/ml bFGF, 20 ng/ml EGF, and B27 supplement in DMEM/F12[Palmer, 1999; Mezey, 2000; Brazelton, 2000]. After 10-14 days, hBMSC showed morphologic and phenotypic characteristics of various neural lineage cells (FIG. 3a). Immunofluorescent cytochemistry revealed expression of phenotypic markers of astrocytes (glial acidic fibrillar protein, GFAP), oligodendrocytes(galactocerebroside, GalC), and neurons(neurofilament 200, NF200; β-tubulin III isoform, β-tubulin III) in 22±7%, 15±6%, 57±9% (NF200) of hBMSC, respectively. RT-PCR confirmed de novo expression of various neural lineage specific genes such as GFAP, myelin basic protein (MBP, oligodendrocyte), MAP2 (microtubule-associated protein 2, neuron), GAD (glutamic acid decarboxylase, neuron), and Tau (neuron) [Sanchez-Ramos, 2000].

To see if hBMSC could differentiate into endodermal lineages, hBMSC were plated at 3-4×10⁴ cells/cm² on 1% Matrigel in 2% FBS, supplemented with 10⁻⁸ dexamethasone, 25 ng/ml HGF, 10 ng/ml FGF-4 and either 10 mg/ml DMSO or 0.5 mM sodium butyrate[Shen, 2000; Hamazaki, 2001; Oh, 2000; Schwartz, 2002]. After 10-14 days of culture, about 60% of hBMSC acquired epithelioid morphology. IF histochemistry revealed expression of endodermal/hepatocytic genes, HNF 3β and αFP at day 10, and HNF 1α and CK18 at day 14 in cultured hBMSC (FIG. 3g-l). RT-PCR analysis revealed de novo expression of endodermal lineage-specific genes, CK18, CK 19, αFP and albumin (FIG. 3m).

FIG. 3A-M are explained in more detail as follows: a. Morphologic characteristics of hBMSC in the neural differentiation culture showing typical appearance of neural cells. scale bar=50 μm. b-e. IF staining of hBMSC after induction of neural differention demonstrates expression of multiple neural specific proteins: GFAP(astrocytes), GalC(oligodendrocytes), NF200 (neurons), β-tubulin III. f. RT-PCR confirms de novo expression of neural lineage-specific genes, GFAP, MBP, oligodendrocyte, MAP2 (neuron), GAD(neuron), and Tau(neuron) after induced neural differentiation. g-l. Phenotypic characterization of hBMSC differentiation into endodermal(epithelioid) cells. IF localization of HNF 3β(FITC) and αFP(Cy3) at day 10 (g-i), and CK18 (FITC) and HNF 1α(Cy3) at day 14 (j-l) in cultured hBMSC on Matrigel with media containing HGF, FGF-4 and DMSO. m. RT-PCR confirms de novo expression of endodermal lineage-specific genes, CK18, CK19, αFP and albumin after induced neural differentiation.

### EXAMPLE 3

### Cardiomyogenic Differentiation of BMSC after Co-Culture with Neonatal Rat Cardiomyocyte (NRCM)

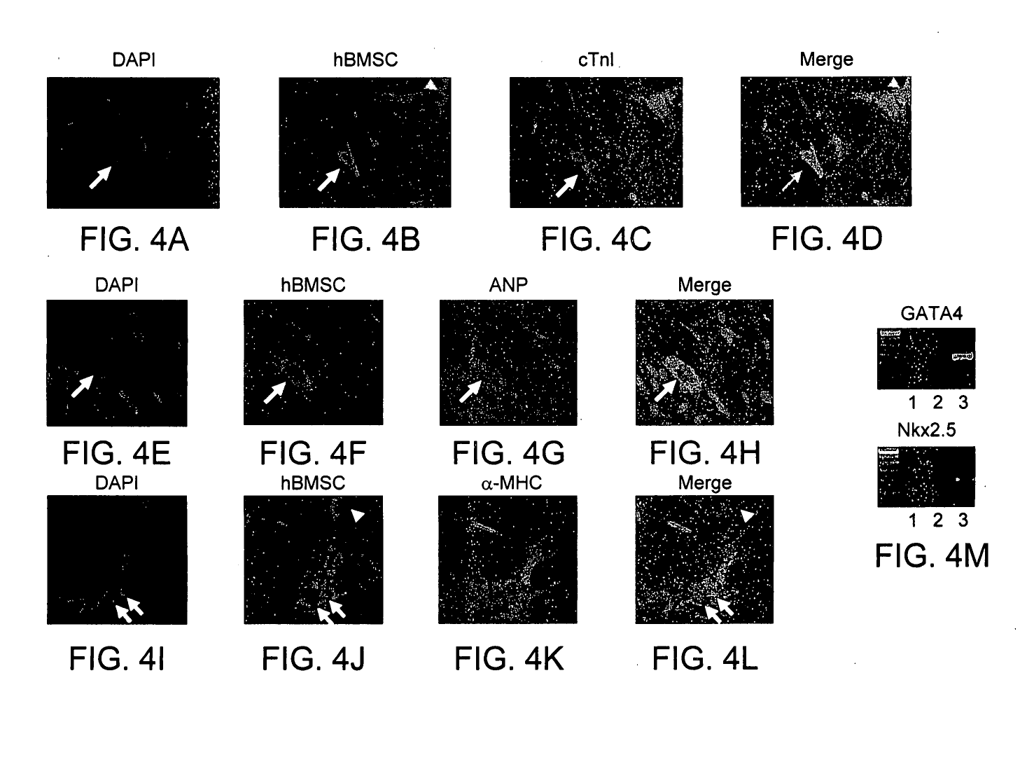

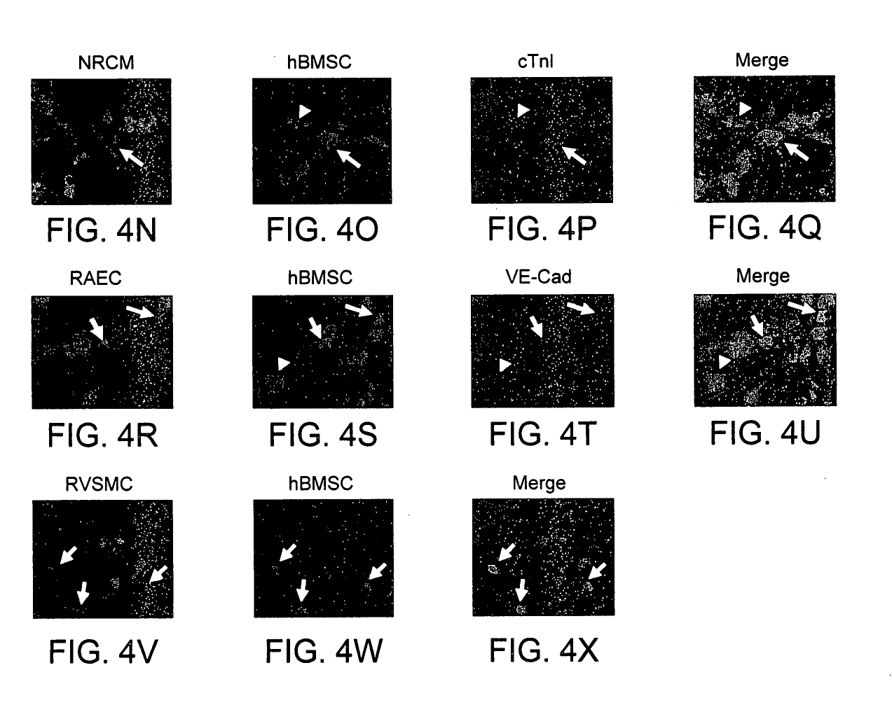

To induce cardiomyogenic differentiation, hBMSC were co-cultured with neonatal rat cardiomyocytes (NRCM). NRCM were plated at 1×10⁵ cells/cm² and cultured in DMEM (low glucose) containing 10% fetal calf serum (FCS). On day 3, hBMSC labeled with DiI were added to the cultured NRCM at a 1:4 ratio [Condorelli, 2001] and cultured up to 2 wks. After fixation and staining with antibodies against cardiac specific markers, immunofluorescent images were obtained. DiI-labeled hBMSC (FIG. 4b, f, j) exhibit red fluorescence from the DiI label and cells positive for CMC specific proteins, such as cardiac troponin I(cTnI), atrial natriuretic peptide(ANP), α-myosin heavy chain (α-MHC) appear green (FIG. 4c, g, k). The merged images illustrate a fraction of DiI-labeled hBMSC which also stained positive for the CMC specific proteins indicating that a subpopulation of hBMSC were displaying features of CMC phenotype in co-culture (FIG. 4). mRNA expression of cardiac transcription factors was evaluated by RT-PCR (FIG. 4m). Before co-culture, GATA-4 and Nkx2.5 were not expressed in hBMSC(left lanes) or NRCM(middle lanes) cultures. Co-culture (right lanes) of hBMSC and NRCM only induced de novo expression of GATA4 and Nkx2.5. GATA-4 is a cardiac specific transcription factor which is known to activate the promoters of several cardiac genes, such as myosin light chain, TnT, TnI and α-MHC, and ANP. Nkx2.5 is a transcription factor restricted to the initial phases of CMC differentiation. [Srivastava, 2000; Bruneau, 2002] Accordingly, these findings suggest that hBMSC have entered a differentiation pathway toward CMC phenotype. Of note, CMC differentiation of hBMSC was not observed when they were co-culture d with conditioned medium from NRCM.

As seen in FIG. 4i, NRCM and hBMSC are tightly associated with each other, thus the possibility of fusion cannot be ruled out by the above experiments. Moreover, recent studies have reported that phenotypic changes of ESCs and bone marrow derived stem cells may occur as a consequence of cell fusion {Ying, 2002} {Terada, 2002) To determine whether this mechanism underlies hBMSC differentiation, CFDA-SE (carboxyfluorescein diacetate succiniimidyl ester)-labeled NRCM and DiI-labeled hBMSC was used for co-culture since neither of these chemical dyes is transferred to adjacent cells. Seven days after co-culture, cultured cells were fixed and stained with cTnI followed by AMCA-conjugated secondary antibody(blue fluorescence) (FIG. 4p). To avoid the overlap of cells, we selected areas of single cell layers for imaging analysis. As shown in FIG. 4n-q certain DiI-labeled hBMSC, marked by an arrow, were also positive for green(NRCM) and blue(cTnI) fluorescence, suggesting that cell fusion had occurred. A truly differentiated cell was illustrated by red and blue fluorescence positive but green fluorescence negative (FIG. 4o-q, arrowhead). These results indicate that both differentiation and fusion occur in the same co-culture conditions. After counting 5000 cells per co-culture from 3 different experiments, the prevalence of differentiation and fusion was 2.9±1.1% and 3.2±0.9%, respectively. To determine the occurrence of fusion between hBMSC and ECs/SMCs, either rat aortic endothelial cells (RAECs) or rat vascular smooth muscle cells (RVSMCs) were co-cultured with hBMSC, respectively. When the RAECs and RVSMCs reached 50-60% confluency, cells were labeled with CFDA-SE (green fluorescence). Two days later, DiI-labeled hBMSC were added to culture plates of RAECs and RVSMCs at a 1:4 ratio and cultured for 7 days. Then, cultured cells were fixed and stained with VE-cadherin or α-SMA followed by AMCA-conjugated secondary antibody. Using the above criteria for fusion and true differentiation under fluorescent microscopy, the prevalence of fusion was 5.3±1.1% for ECs and 7.4±0.9% for SMCs, while the prevalence of differentiation was 3.4±0.7% and 3.6±0.5%. These results disclosed that phenotypic changes of hBMSC consist of both fusion and differentiation, and therefore, immunohistochemistry using lineage-specific marker proteins for defining differentiation may overestimate the true prevalence of differentiation in vivo. It must be acknowledged that the rates of fusion and differentiation in vivo may differ from those documented in vitro.

FIGS. 4A-M are explained more as follows. a-m. Co-culture of hBMSC with NRCM for investigation of cardiomyogenic differentiation. Primary NRCM from F344 rats were isolated and cultured in DMEM. On day 4, DiI-labeled hBMSC were added to the cultured NRCM at a 1:4 ratio and cultured up to 2 wks. IF images show co-cultured DiI-labeled hBMSC(b, f, j) as red and NRCM stained with CMC specific proteins, cTnI (c), ANP(g) and α-MHC(k) as green. Double fluorescence positive cells in the merged images (d, h, l) indicate that a subpopulation of hBMSC exhibit CMC phenotypes(arrows). Note that a few hBMSC remain non-transdifferentiated (arrowheads in b, d, j, l). DAPI nuclear counter-staining(a, e, i) shows no overlap of nuclei suggesting this apparent trans-differentiation does not result from a mere overlap of hBMSC and NRCMs. mRNA expression of cardiac transcription factors was evaluated by RT-PCR (m). Before co-culture, cardiac transcription factors, GATA4 and Nkx2.5 were not expressed in hBMSC(lane 1) and NRCM(lane 2) cultures. Co-culture (lane 3) induced de novo expression of GATA4 and Nkx2.5. n-q. Co-culture of pre-labeled NRCM and hBMSC for investigation of cell fusion. To determine whether fusion of both cells may underlie the change into CMC phenotypes, we co-culture d NRCMs labeled with green fluorescence dye, DFCA-SE (n) and DiI-labeled hBMSC (o). Seven days after co-culture, cells were stained with cTnI followed by AMCA-conjugated secondary antibody (blue fluorescence)(p). In the IF images (n-q), triple color positive cells (arrow) are fusion cells expressing cTnI protein. A hBMSC which has transdifferentiated into CMC lineage is illustrated by red and blue fluorescence positive but green negative (arrowhead). r-x. To determine the contribution of fusion and differentiation to phenotypic changes of hBMSC into ECs (r-u) or SMCs (v-y), CDFA-SE-labeled rat aortic endothelial cells (RAECs) or rat vascular smooth muscle cells (RVSMCs) were co-cultured with DiI-labeled hBMSC at a 4:1 ratio and cultured for 7 days. After fixation, cells were stained with VE-Cadherin or α-SMA followed by AMCA-conjugated secondary antibody(blue fluorescence). Arrow in r-u indicates fusion between RAECs(green) and hBMSC(red) which expresses VE-Cadherin. Arrowheads in r-u illustrate ECs differentiated from hBMSC. Arrow in v-x indicates fusion between RVSMCs(green) and hBMSC(red).

### EXAMPLE 4

### Regenerative Effect on Acute Ischemic Myocardium

We tested whether ischemically jeopardized myocardium could be restored by transplantion of hBMSC as follows.

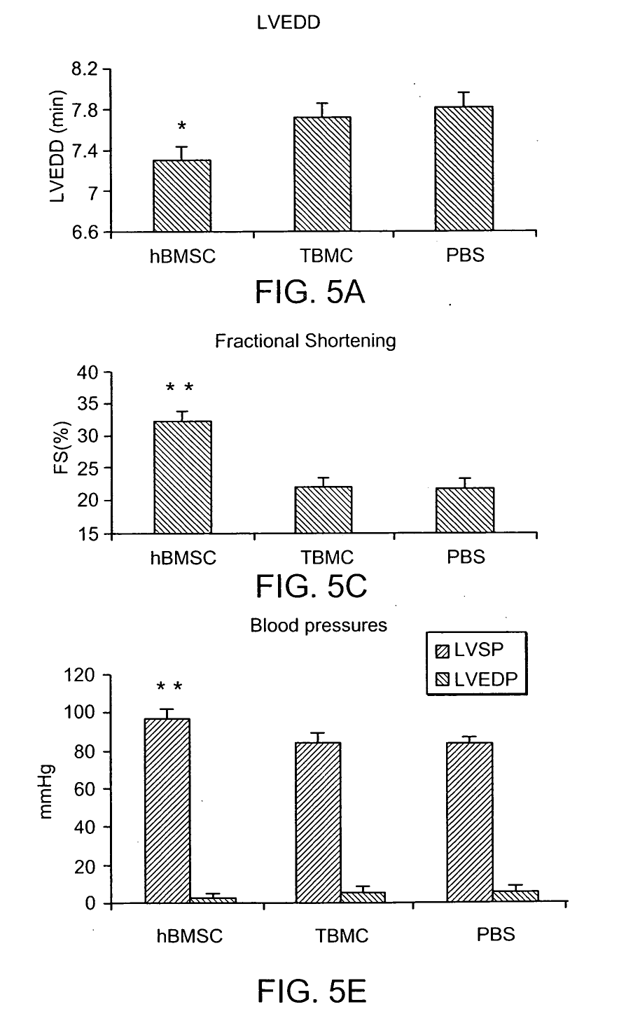

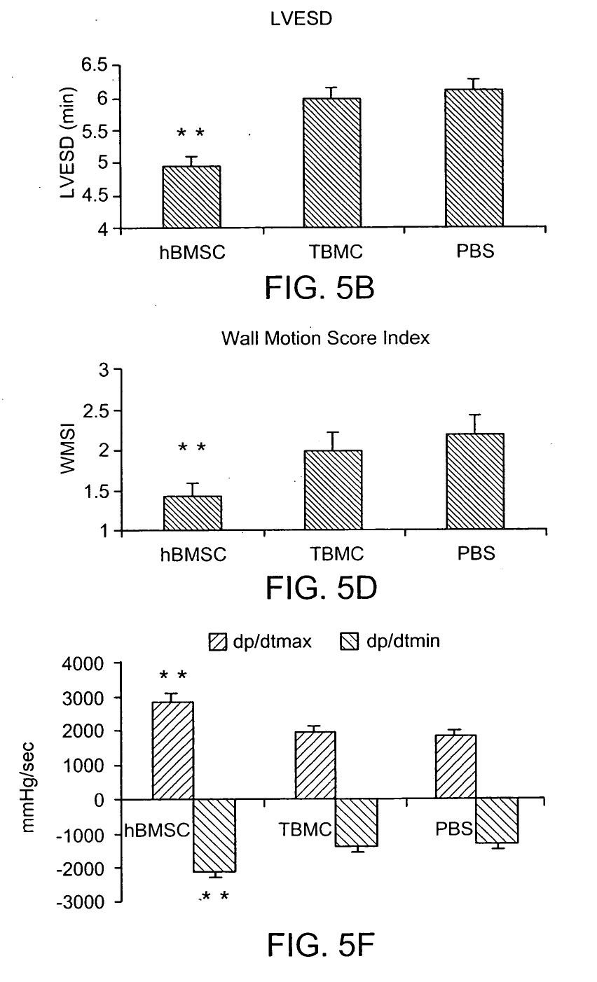

Shortly after coronary ligation in nude rats, 8×10⁵ hBMSC were transplanted in the wall of peri-infarct area. The same number of human total bone marrow cells (TBMC) and the same volume of PBS were used as controls. Out of 15 rats that underwent surgery, 14 rats receiving hBMSC survived while 12 rats survived in TBMC and PBS groups during the study period of 4 wks. To evaluate the cardiac function, echocardiography was performed before and 4 wks after the surgery. Baseline left ventricular end-diastolic and end-systolic dimensions (LVEDD, LVESD) fractional shortening (FS) and wall motion score index (WMSI) were similar among rats receiving hBMSC, TBMC or PBS(data not shown). Echocardiography performed 4 wks after treatment revealed that, both LVEDD and LVESD were significantly smaller (LVEDD, P<0.05 versus TBMC and PBS, respectively; LVESD, P<0.01 versus TBM and PBS, respectively) (FIG. 5a,b) and thus FS was significantly greater (P<0.01 versus TBMC and PBS, respectively) (FIG. 5c) in rats receiving hBMSC compared with those treated with TBMC or PBS. Wall motion score index (WMSI), which represents the extent of regional wall motion abnormalities, was significantly better (P<0.01 versus TBMC and PBS, respectively) (FIG. 5d). LVEDD, LVESD, FS, and WMSI were not different between TBM and PBS treated rats. Of note, the incidence of dyskinesia was 14% vs. 58% and 67% in hBMSC vs. TBMC and PBS treated rats (P<0.05). Invasive hemodynamic measurements demonstrated LV systolic pressure (LVSP), +dP/dt (maximal rates of pressure rise) and −dP/dt (maximal rate of pressure fall) were all significantly greater in the hBMSC transplanted rats than in the TBMC or PBS injected rats (all P<0.01, respectively) (FIG. 5e,f). Together, these data indicate that hBMSC transplantation results in enhanced functional recovery and more favorable remodeling following myocardial infarction.

FIG. 5A-D is explained more fully as follows. a-d. Echocardiographic parameters 4 wks after MI and cell transplantation show smaller LVEDD, LVESD and better FS and WMSI in the hBMSC transplanted rats compared to the TBMC and PBS treated rats, indicating improved cardiac function. WMSI: Wall motion score index. e, f. Invasive hemodynamic measurements using Millar catheter 4 wks after the hBMSC transplantation. LV systolic pressure (e) and +dP/dt and −dP/dt were significantly augmented in the hBMSC transplanted rats compared to the control groups'. *P<0.05, **P<0.01.

### EXAMPLE 5

### Engraftment and Multi-Lineage Trans-Differentiation of Transplanted hBMSC in Vivo

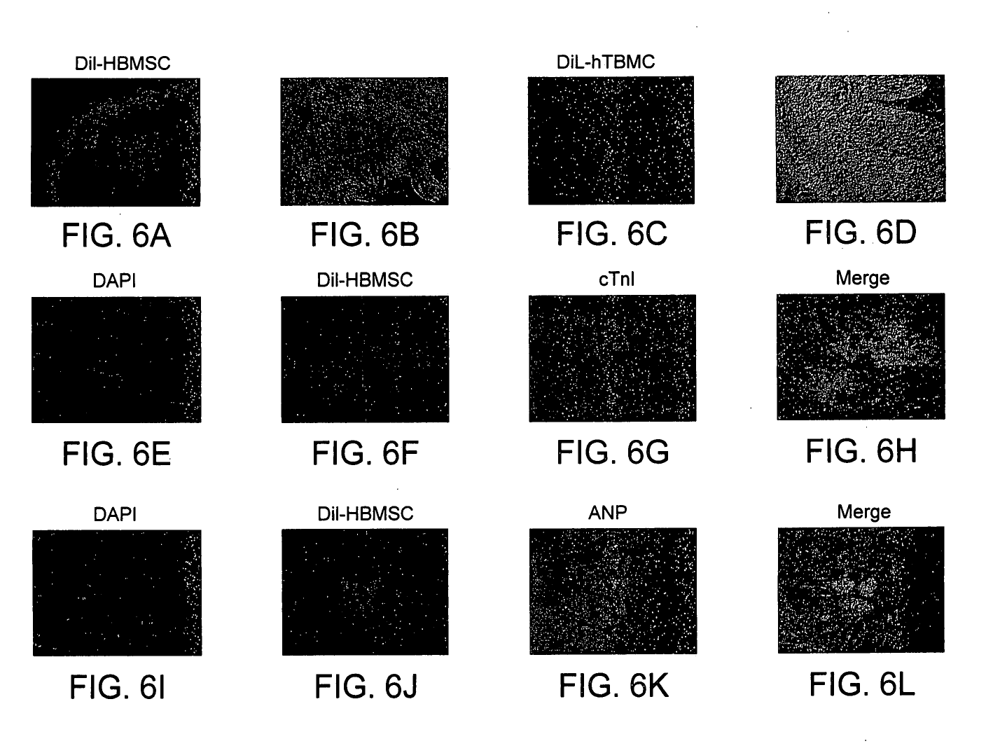

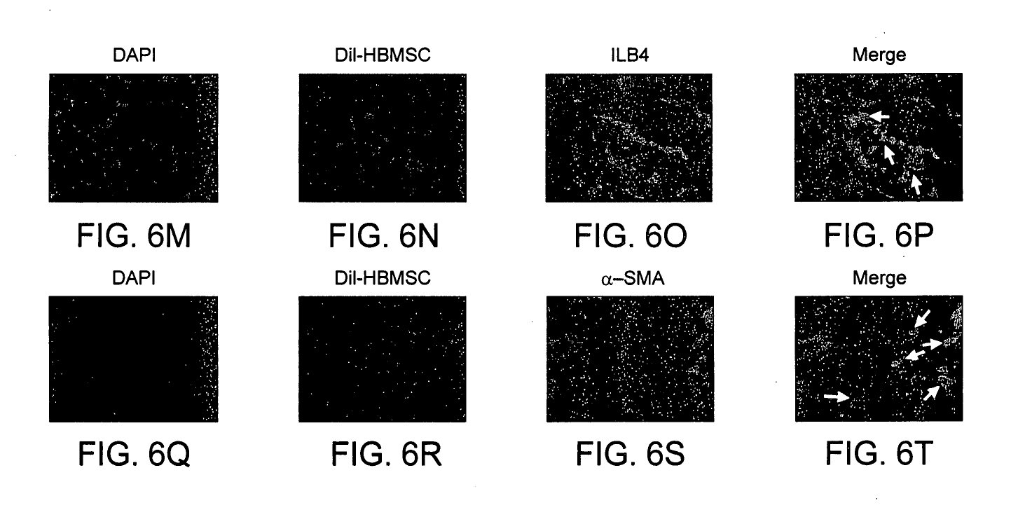

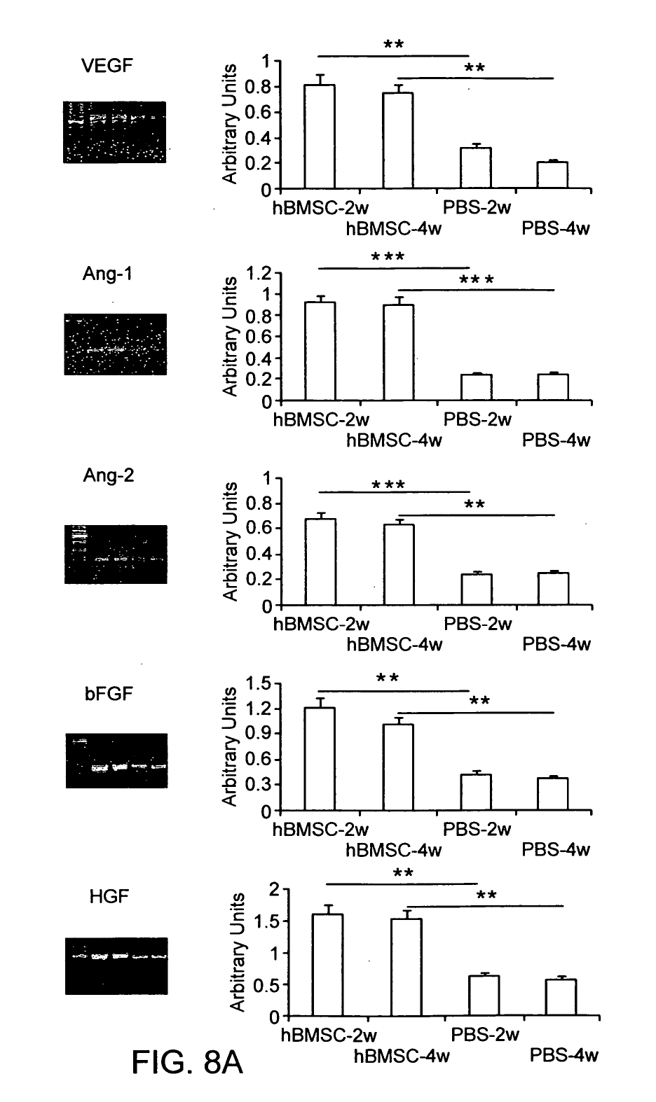

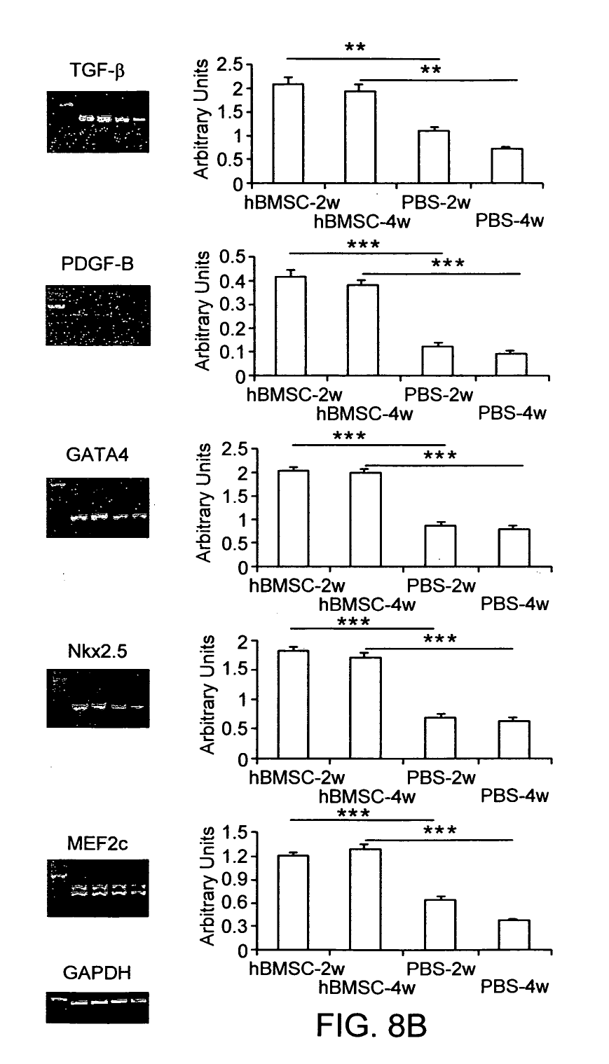

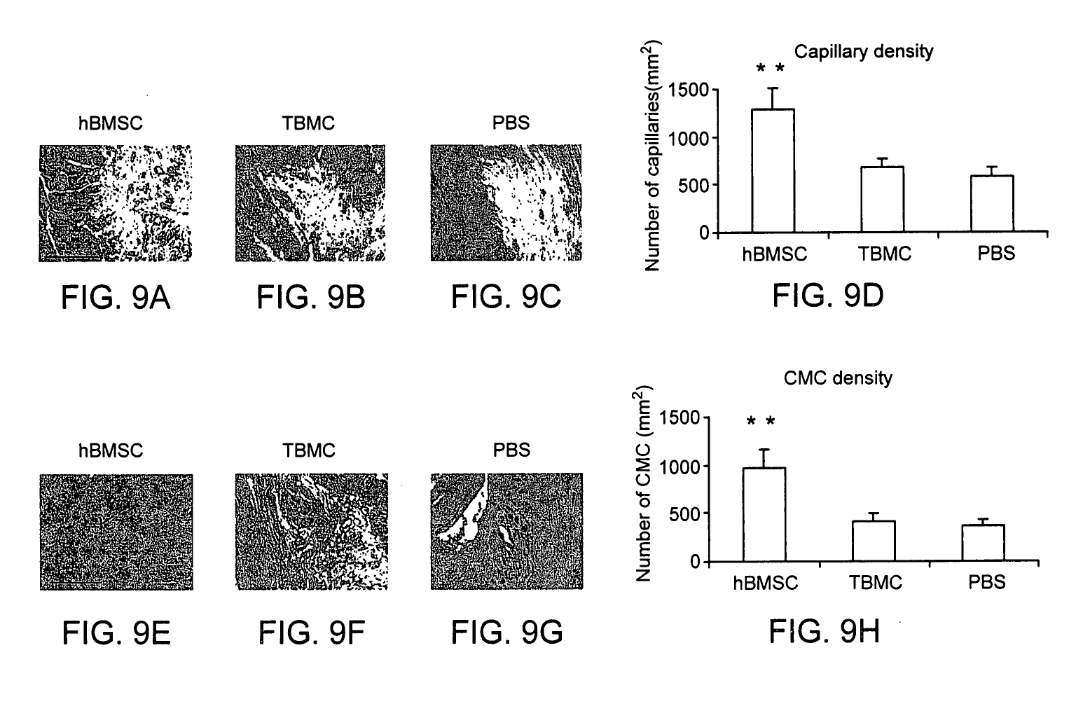

To determine the extent and magnitude of transplanted hBMSC engraftment, 4 wk-myocardial sections from frozen samples were examined. Under fluorescent microscopy, engraftment of numerous DiI-labeled BMSCs in the peri-infarct and infarct region was observed (FIG. 6a,b). In contrast, the TBMC transplanted heart shows smaller numbers of scattered DiI-labeled cells, which are mostly located within the infarct region (FIG. 5c, d). Immunophenotypic characterization revealed that engrafted DiI-labeled hBMSC(red) were stained positive for CMC specific proteins, cTnI and ANP(green)(α-MHC data not shown) and formed a mass of morphologically mature appearing CMCs, indistinguishable from host CMCs in the periinfarct and infarct area (FIG. 6e-l), indicative of CMC differentiation. Differentiation of hBMSC into ECs and SMCs was also confirmed by the co-localization of transplanted hBMSC with ILB4 positive vascular ECs and α-SMA positive SMCs, suggestive of differentiation into EC and SMC phenotypes (FIG. 6m-t). CMC differentiation only in hBMSC transplanted rats. Differentiation into CMC, defined by morphologic similarity and CMC specific protein expression (cTnI), was observed in 12 out of 14 surviving hBMSC transplanted rats (85%) could be detected. An organized mass of regenerating CMCs similar to normal host myocardium as seen in FIG. 6h and FIG. 8c was found in 9 out of 14 rats (64%) and only scattered cellular differentiation was observed in 3 other rats (21%). However, CMC specific protein expression was observed in all hBMSC treated rats. EC and SMC differentiation defined by morphology and marker positivity was found in all rats examined. Together, these findings suggested that transplanted hBMSC were robustly engrafted in the infarct/ischemic myocardium, survived for prolonged periods of time and resulted in de novo myogenesis and vasculogenesis. Of note, pathologic investigation revealed no teratoma, angiomatosis or bone formation in hBMSC transplanted hearts.

FIG. 6A-D are explained in more detail as follows. Engraftment of DiI-labeled hBMSC and TBMC into infarcted myocardium. Numerous hBMSC(red fluorescence)(a) are engrafted into infarct and peri-infarct region of myocardium at 4 wks after transplantation. In contrast, considerably fewer TBMCs(red fluorescence) are seen, mostly within the infarct area(c). (b) and (d) are the Hoffman images of (a) and (c) showing the localization of engrafted cells. e-l. Immunophenotypic characterization of differentiated hBMSC into CMCs. Four wk myocardial samples were stained for cTnI(g) and ANP(k)(each detected with FITC-labeled secondary Ab). Transplanted DiI-hBMSC were stained positive for both markers, and indistinguishable from host CMCs, indicative of regeneration of a mass of CMCs. m-p. Incorporation of hBMSC into vascular ECs. Myocardial sections stained with ILB4 (o), an EC marker, demonstrated DiI-hBMSC are co-localized with vascular ECs suggestive of trans-differentiation into ECs(arrows, p). q-t. Incorporation of hBMSC into vascular SMCs. Myocardial sections stained with α-SMA(s) illustrate DiI-hBMSC co-localized with vascular SMCs, indicative of differentiation into SMCs phenotypes (arrows, t).

### EXAMPLE 6

### Transplanted hBMSC Augmented Proliferation and Survival of Host Myocardium

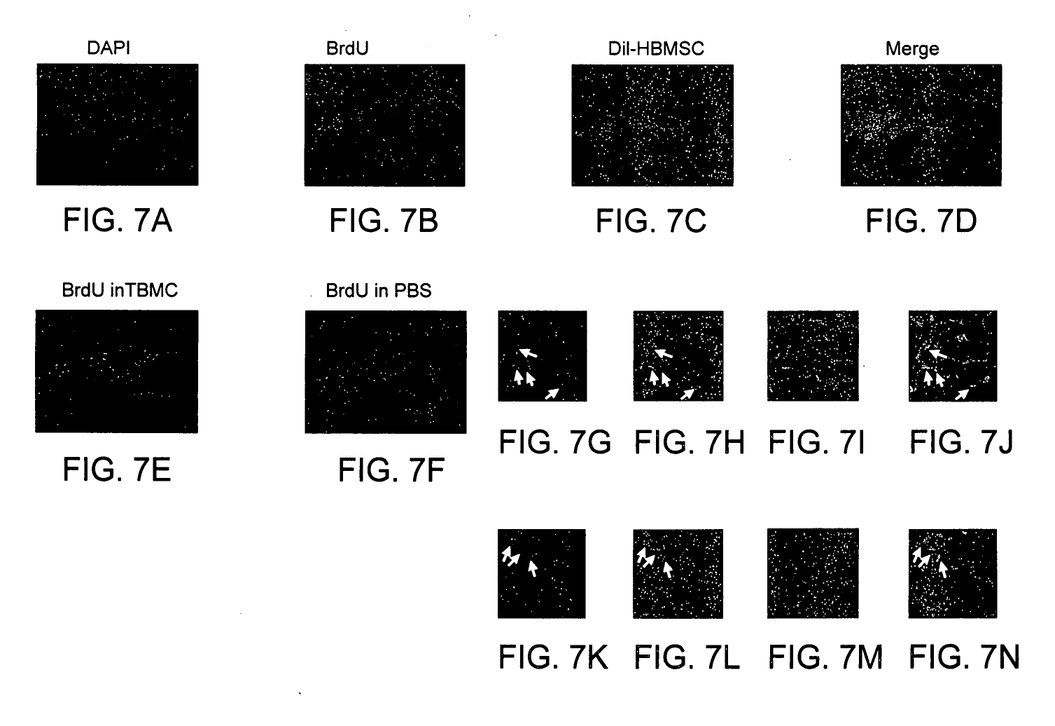

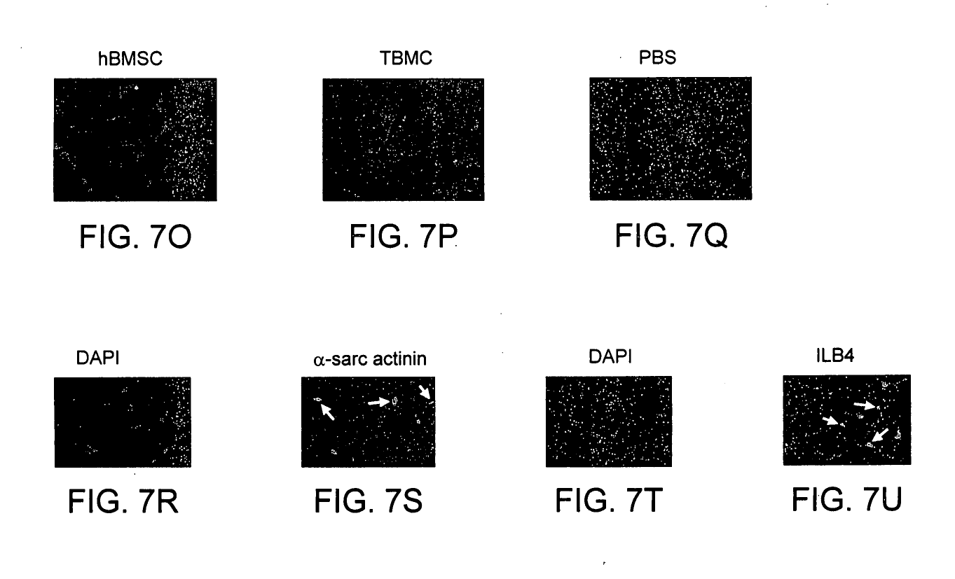

Since the profound physiologic effect of hBMSC transplantation may not solely be explained in some of the rats by the extent of multilineage differentiation in situ, we next sought to elucidate whether transplanted hBMSC affected proliferation and survival of host myocardial cells. To determine the proliferative fraction of host myocardial cells, mini-osmotic pumps were implanted for delivering 5-Bromo-2′-deoxyuridine(BrdU) constantly for 4 wks to label all cells that entered the S phase of the cell cycle. In sections from hBMSC transplanted hearts, numerous proliferating cells were observed both in host myocardial cells and transplanted cells (FIG. 7a-d). In contrast, considerably fewer BrdU-positive cells were observed in TBMC or PBS injected hearts (FIG. 7e,f). A BrdU index, the percentage of the BrdU positive nuclei to total number of nuclei counted in each section was significantly higher in hBMSC transplanted hearts than controls (hBMSC vs. TBMC, PBS; 25.8±5.2% vs. 11.2±3.1%, 9.5±2.8%, P<0.001). Double IF histochemistry using mAb against BrdU and either ILB4 or cTnI revealed that CMCs and ECs were positive for BrdU (FIG. 7g-n). The BrdU indices of ECs and CMCs were again higher in hBMSC transplanted hearts (vs. TBMC, PBS; ECs, 8.3±2.7 vs. 2.9±1.5, 2.4±1.3, P<0.01; CMCs, 2.1±1.2 vs. 0.2±0.1, 0.2±0.1, P<0.01). These results indicate that the transplanted hBMSC augment proliferation of host myocardial cells including ECs and CMCs.

Next, we investigated whether transplanted cells could affect the apoptotic process, generally regarded as one of the main mechanisms responsible for ongoing myocardial degeneration following acute MI. TUNEL assays were performed using 1 wk myocardial samples (n=4). The apoptotic index, the percentage of TUNEL(+) nuclei to total number of nuclei counted in each section, was 3 times lower in hBMSC transplanted hearts (apoptotic index vs. TBMC, PBS, 2.1±0.8 vs. 6.6±1.2, 7.3±1.1, P<0.01) (FIG. 7o-q). These differences were particularly evident within the periinfarct area. The apoptotic index of CMCs and ECs obtained after concomitant staining of α-sarcomeric actinin or ILB4 and TUNEL assay, was significantly decreased in hBMSC transplanted hearts (vs. TBMC, PBS; CMCs, 0.5±0.1 vs. 2.1±0.3, 1.9±0.3, P<0.01; ECs, 0.7±0.1 vs. 2.5±0.3, 2.0±0.2, P<0.01) (FIG. 7r-u). Thus, an alternative mechanism by which hBMSC transplantation can preserve myocardial function, promoting survival of endangered CMCs and ECs, is elucidated by these studies.

FIG. 7A-U is explained more fully as follows. a-h. Identification of proliferative cells by BrdU imunohistochemistry. In sections from hBMSC transplanted heart (a-d), numerous BrdU positive cells (green dot) were observed (c) both in host myocardial cells and transplanted cells. In contrast, fewer BrdU positive cells were observed in TBMC(e) or PBS(f) injected rat hearts. g-j. Concomitant staining with antibody against BrdU(h) and ILB4 (i) demonstrates the presence of BrdU positive cells (arrows) in capillary ECs of a hBMSC transplanted heart. k-o. Concomitant staining with antibody against BrdU(I) and cTnI(m) shows the presence of BrdU positive cells (arrows) in mature CMCs(n) of a hBMSC transplanted heart. m-o. Concomitant staining of hBMSC(m), TBMC(n) or PBS(o)-treated myocardium with anti-α-sarcomeric actinin mAb and TUNEL to identify apoptotic myocardial cells (arrows). Fewer apoptotic cells are evident in the peri-infarct area of rats receiving hBMSC(o) compared to those receiving TBMC(p) or PBS(q). Magnification×400. r-s. A representative image obtained after staining with mAb against α-sarcomeric actinin (red) and TUNEL (green) demonstrates CMC apoptosis (arrows) from a PBS injected heart. t-u. A representative image obtained after staining with ILB4 (red) and TUNEL (green) demonstrates EC apoptosis (arrows) from a PBS injected heart.

### EXAMPLE 7

### Paracrine Effect of Transplanted hBMSC: Upregulation of Angiogenic Cytokines and Cardiac Transcription Factors

To identify potential paracrine mechanisms responsible for the therapeutic effect of hBMSC following myocardial injury, mRNA expression of angiogenic cytokines such as VEGF-A, angiopoietin-1(Ang-1) and 2 (Ang-2), hepatocyte growth factor(HGF), basic fibroblast growth factor(bFGF), platelet-derived growth factor-B(PDGF-B), transforming growth factor(TGF)-β and cardiac transcription factors such as Nkx2.5, GATA-4, MEF2c by semi-quantitative RT-PCR using harvested cardiac samples at 14 and 28 days after hBMSC or PBS treatment was evaluated. Expression of all the examined angiogenic cytokines was significantly upregulated both at 2 and 4 wks in the hBMSC transplanted hearts compared to the PBS group (P<0.01). Moreover, the high expression of all the angiogenic cytokines was maintained at 4 wks in the hBMSC transplanted hearts. These findings suggest the transplanted hBMSC can modulate expression of multiple angiogenic cytokines with the potential to promote angiogenesis as well as vasculogenesis. GATA-4, Nkx2.5 and MEF2c were significantly upregulated in the hBMSC transplanted hearts compared to the controls (FIG. 8b), suggesting that transplanted hBMSC could enhance myogenesis by augmenting host CMC differentiation and/or directly differentiating into new CMCs. Collectively, the upregulation of multiple factors can, in part, explain the proliferative effect of transplanted hBMSC on host ECs and CMCs.

FIG. 8A-B are explained as follows. Tissue samples were harvested at 2 and 4 wks after hBMSC or PBS treatment and mRNA expression was examined by semi-quantitative RT-PCR. Representative gel photographs (n=4) (left panels) of RT-PCR product, and quantification of mRNA expression (right panels) of angiogenic cytokines(a) and cardiac transcription factors(b) based on the GAPDH expression. Note the significant upregulation of all factors in the hBMSC transplanted samples. Experiments was performed at least triplicates. **P<0.01, ***<0.001

### EXAMPLE 8

### hBMSC Transplantation Increased Capillary and CMC Density and Decreased Myocardial Fibrosis

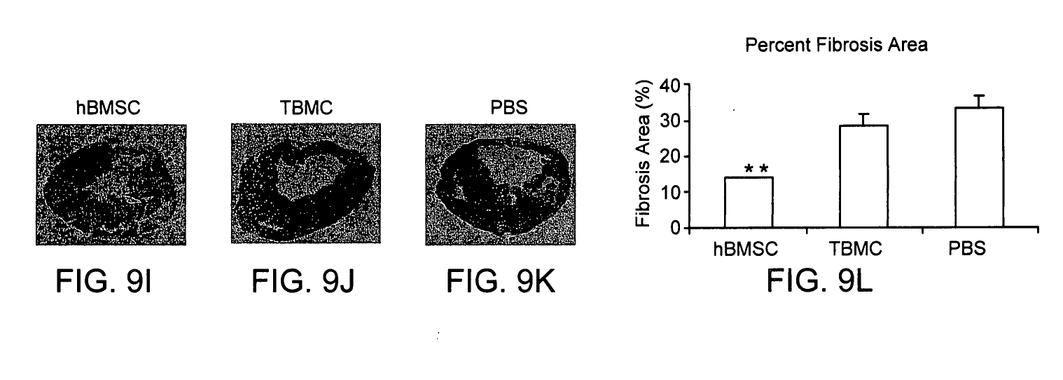

To determine the final impact of hBMSC transplantation on the pathologic features of infarcted myocardium, capillary and CMC density were quantified after CD31 and HE staining, respectively and the percent circumferential fibrosis area was measured. Capillary density was 2 and 2.3 fold higher in the hBMSC transplanted rats than in the TBMC and PBS injected rats (P<0.01) (FIG. 9a-d). CMC density was also 2.4 and 2.8 fold higher in the hBMSC transplanted rats than in the TBMC and PBS injected rats (P<0.01) (FIG. 9d-g). Percent circumferential fibrosis area measured in Masson's trichrome-stained sections demonstrated a smaller area of fibrosis (blue colored area) in the hBMSC transplanted hearts (P<0.01 vs. TBMC, PBS).

FIG. 9A-G are explained in more detail as follows. a-d. Representative immunohistochemical findings for CD31(a-c) in infarct myocardium 4 wks after cell transplantation showing significantly higher capillary density in a hBMSC transplanted heart. **P<0.01 vs. TBMC and PBS. e-h Representative findings of infarct area stained with HE(e-g) demonstrates notable salvage and regeneration of myocardium in a hBMSC transplanted heart. **P<0.01 vs. TBMC and PBS. i-l. Representative figures of Masson's trichrome-stained hearts showing significantly smaller area of percent fibrosis in a hBMSC transplanted heart. **P<0.01 vs. TBMC and PBS.

The following materials and methods were used as needed to conduct the experiments shown above as Examples 1-8.

1. Isolation and Culture of BMSC

Fresh unprocessed human BMs from young male donors were purchased from Biowhitttaker (Cambrex) (Wakersville, Md.). BM was centrifuged at 1300 rpm for 10 minutes to obtain cell pellets. Cell pellets were resuspended in 25 ml of DPBS containing 0.5M EDTA(DPBS-E). After centrifugation at 1300 rpm for 7 minutes, cells were resuspended in 5 ml DPBS-E and 20 ml of NH₄Cl for induction of hemolysis. After centrifugation and washing with DPBS-E, cells were filtered through a 40-μm Nylon filter and were plated in wells of 6-well plates that had been coated with fibronectin (100 μg/ml). The cells were grown in complete DMEM with low (1 g) glucose containing 17% of FBS (Biowhittaker; lot selected for promoting expansion of marrow cells), 100 U/ml penicillin, and 100 μg/ml streptomycin and 2 mM glutamate at 37° C. and 5% CO² for 4-6 days, the medium was replaced with fresh complete medium, and the adherent cells were grown to 60% confluency. Next, the cells were reseeded in complete medium into 25 cm² tissue culture flask (T25) at a density of 1×10⁴ cells/cm². After the cells were 60% confluent, the cells were serially reseeded into T75 and T175 flasks at the same density. After at least 2 passages of cultures in T175 flasks, cells were labeled with CellTracker™ CM-DiI (Molecular probes) and plated into wells of 96-well plate at a density of ½ cell per well by limiting dilution method and cultured with conditioned media which were collected before limiting dilution, store in −80° C. and filtered through a 0.2 μm filter. Under the fluorescent microscopy, we excluded wells containing more than one cell (FIG. 1a) shows the phase contrast and fluorescent image of a single cell in a well. When cells were grown to 40-50% confluence, cells from one well were serially reseeded into one well of 6-well plates and thereafter serially reseeded in T25, T75 and T175 at a density of 4-8×10³ cells/cm². Cells were then cultured in T175 and replated 1:10-40 dilution and were grown to 4-8×10³ cells/cm².

2. Fluorescent-Activated Cell Sorting

Fluorescent-activated cell sorting (FACS) analysis of hBMSC was performed on cultured hBMSC. Cells from at least 3 different clones of pre- and post-clonal isolation were used, and for clonal lines, two sets of cells at 5 PDs and 120 PDs were used. The procedure of FACS staining was described previously {Kalka, 2000 #11}. In brief, a total of 2×10⁵ cultured cells were resuspended with 200 μl of Dulbecco's PBS (Cambrex) containing 10% FBS and 0.01% NaN₃ and incubated for 20 minutes at 4° C. with directly PE- or FITC-conjugated monoclonal antibodies or non-conjugated antibodies followed by a FITC-conjugated rabbit anti-mouse IgG (Jackson Immunoresearch). Proper isotype-identical immunoglobins served as controls (Beckton-Dickinson, Medford, Mass., USA). After staining, the cells were fixed in 2% paraformaldehyde. Quantitative FACS was performed on a FACStar flow cytometer (B-D). Antibodies used for FACS analysis were FITC- or Phycoerythrin (PE)-conjugated antibodies against CD4, CD8, CD11b(Mac-1), CD13, CD14, CD15, CD29, CD30, CD31, CD34, CD44, CD71, CD73, CD90(Thy1), CD117(c-kit), CD146, CD166, HLA-DR, HLA-ABC, β2-microglobulin from Beckton Dickson(BD), CD133(AC133) from Miltenyl Biotech (Auburn, Calif., USA), and CD105(Endoglin) from Ancell (Bayport, Minn., USA), and unconjugated antibodies against Oct4 from Santa-Cruz. For comparison, human mesenchymal stem cells (PT-2501) and media (PT-3001) were purchased from Cambrex (Poietics) and used for FACS.

3. DNA Ploidy Analysis

DNA contents per cells were determined by pre-treating cells with Ribonuclease (100 μg/ml) and staining with propidium iodide (50 μg/ml) and subsequent FACS analysis.

4. Telomere Length Assay

We used TeloTAGGG Telomere Length Assay kit (Roche, Indianapolis, Ind.) for determination of telomere length of hBMSC at 5 PDs and 120 PDs from 3 different clones. Briefly, after isolation (1 μg) and digestion of genomic DNA, DNA fragments were separated by gel electrophoresis and transferred to a Nylon membrane by Southern blotting. The blotted DNA fragments are hybridized to a digoxigenin (DIG)-labeled probe specific for telomeric restriction fragment (TRF) and incubated with a DIG-specific ab covalently coupled to alkaline phosphate. Finally, the immobilized telomere probe is visualized by virtue of alkaline phosphatase metabolizing CDP-Star, a highly sensitive chemiluminescence substrate. The average TRF length was determined by comparing the signals relative to a molecular weight standard.

5. In Vitro Differentiation of hBMSC

All of the following in vitro studies were performed by using 3 different clonal hBMSC of 5 and 90 PDs. To induce differentiation into ECs, hBMSC were replated at 5×10⁴ cells/cm² in either 0.1% gelatin or vitronectin coated glass chamber slides in DMEM or EBM-2(Clonetics) with 2-5% FBS, 10⁻⁸ dexamethasone and 10 ng/ml VEGF (R&D, Minneapolis, Minn.) and cultured up to 14 days. To induce differentiation into SMC phenotypes, hBMSC were replated at 1×10⁵ cells/cm² in non-coated or fibronectin coated culture dishes in 1-2% DMEM or EBM-2 supplemented with PDGF-BB (50 ng/ml, R&D) [Hellstrom, 1999 #756; Yamashita, 2000 #757] and cultured for 14 days. To induce neural lineage differentiation, hBMSC were plated at 4×10⁴/cm² on Poly-L-ornithin-laminin coated dishes (B-D) or plastic dishes with 100 ng/ml bFGF and 20 ng/ml EGF, B27 supplement in DMEM/F12 [Palmer, 1999 #764; Mezey, 2000 #750; Brazelton, 2000 #749] and cultured up to 14 days. To induce endodennal lineage differentiation, hBMSC were plated at 3-5×10⁴ cells/cm² on 1% matrigel (BD) in 2% FBS, supplemented with 10⁻⁸ dexamethasone, 25 ng/ml HGF, 10 ng/ml FGF-4 and cultured for 7 days, and thereafter either 10 mg/ml DMSO or 0.5 mM sodium butyrate were added and cultured for 7 more days [Shen, 2000 #761; Hamazaki, 2001 #762; Oh, 2000 #763; Schwartz, 2002 #747]. To induce CMC differentiation of the hBMSC, hBMSC were co-cultured with freshly isolated NRCM. Primary NRCM from F344 rats were isolated as described [De Luca, 2000 #759] and plated at 2×10⁵ cells/cm². DiI-labeled hBMSC were added to the NRCM culture plates at a 1:4 ratio in DMEM containing 10% fetal calf serum (FCS) [Condorelli, 2001 #758] and cultured up to 10 days.

6. In Vitro Fusion Studies

Rat aortic endothelial cells (RAECs) were cultured in EBM-2 containing EGM-2 MV Single Quots (Cambrex). Rat vascular smooth muscle cells (RVSMCs) were cultured in DMEM with 1 g/L glucose (Cellgro) containing 15% FBS, 2 mM L-glutamine and antibiotics. NRCMs were isolated and cultured as described above. When the cells reached 50-60% confluency (within 3 days), cells were stained with 25 μM of Vybrant CFDA SE cell tracer kit (Molecular Probes, Engene, Oreg.) according to the manufacturer's instruction. Cells labeled with CFDA SE is shown green under the fluorescent microscopy. Two days later, DiI-labeled hBMSC (red fluorescence) were added to culture plates of RAECs, RVSMCs or NRCM at a 1:4 ratio and cultured for 7 days. Media were changed every 2-4 days. To decide the prevalence of fusion and differentiation, 5000 cells were counted per co-culture from 3 different experiments.

7. Antibodies Used for Immunofluorescent Cytochemistry

For immunocytochemistry, cells were fixed in 4% cold paraformaldehyde for 7 minutes and washed with PBS twice. As EC markers, we used abs against vWF(goat pAb)(1:400, Sigma, St Louis, Mo.), Flk-1(mouse mAb)(1:300, Santa-Cruz, Santa-Cruz, Calif.) VE-Cadherin(mouse mAb)(1:100, BD), CD31(mouse mAb)(1:100, BD), UEA-1 lectin (1:200, Vector, Burlingame, Calif.), isolectin B4(1:200, Vector) and DiI-acetylated LDL(Biomedical Technologies, Stoughton, Mass.). As SMC markers, we used abs against α-smooth muscle actin(mouse mAb)(1:300), calponin(mouse mAb)(1:250, DAKO, Carpinteria, Calif., USA). As neural lineage markers, we used abs against NF-200(mouse mAb)(1:400, Sigma), β-tubulinIII(mouse mAb)(1:100, Sigma), Gal-C(rabbit pAb)(1:100, Sigma), GFAP(goat pAb)(1:200, Santa-Cruz). As endodermal (hepatocytic or other epithelial) lineage markers, we used CK18 (mouse mAb; 1:300)(Sigma), α-fetoprotein (goat pAb, 1:200)(Santa-Cruz), albumin (mouse mAb, 1:400)(Sigma), HNF3β (goat pAb, 1:100)(Santa-Cruz), HNFα (rabbit pAb, 1:200)(Santa-Cruz). As CMC markers, abs against cTnI (two forms: mouse mAb and rabbit pAb, 1:100) (Chemicon, Temecular, Calif.), ventricular myosin heavy chain α(α-MHC)(mouse mAb, 1:100)(Chemicon), α-sarcomeric actinin (clone EA-53, mouse mAb, 1:200)(Sigma), ANP(rabbit pAb)(Chemicon). Control mouse, rabbit or goat IgG were from Sigma. Secondary anti-mouse, rabbit or goat antibodies (AMCA, 1:150, FITC or Cy-2, 1:200; Cy-3, 1:200) were from Jackson Immunoresearch (West Grove, Pa.).

8. Study Design of In Vivo Experiments

All procedures were performed in accordance with the Caritas St. Elizabeth's Institutional Animal Care and Use Committee. Female nude rats (Hsd: RH-mu rats, Harlan, Indianapolis, Ind.) of 6-7 wks-old underwent surgery to create acute MI, followed immediately by intramyocardial injection of 8×10⁵ hBMSC or 8×10⁵ human fresh total BM cells (TBMC) or PBS in a total volume of 200 μl at 5 sites (basal anterior, mid anterior, mid-lateral, apical anterior, apical lateral) in the periinfarct area. The rats were randomly assigned to each group of 15. The preparation of TBMCs was the same as that of hBMSC before culture. hBMSC and TBMCs were labeled with carbocyanine dye, DiI(1.5 μg/ml, Molecular Probes) before the cell transplantation as described before [Kalka, 2000; Kawamoto, 2001]. Separate rats (n=4, each group) were implanted underneath the back skin with miniosmotic pumps (model 2ML4, Alzet, Palo Alto, Calif.) that infused 1.25 mg BrdU/d (Sigma) (dissolved in a 1:1 (vol/vol) mixture of dimethyl sulfoxide and 0.154 M NaCl) after the surgery and sacrificed at 4 wks {McEwan, 1998}. For TUENL assay and molecular studies, another rats were similarly operated and sacrificed 1 and 2 wks after surgery (n=4, each time points). Following surgery, rats underwent echocardiography (Echo) and invasive hemodynamic measurements 28 days later. The rats were then, sacrificed with saline perfusion into the aorta. At necropsy, hearts were sliced into 3 transverse sections from apex to base, fixed with 4% paraformaldehyde, methanol, or frozen in OCT compound and sectioned with 5 μm thickness.

9. Acute Myocardial Infarction Model

MI was induced as described previously in our lab [Kawamoto, 2003; Kawamoto, 2001]. Following left thoracotomy and incision of the pericardium under artificial ventilation (model 683, Harvard Apparatus), the left anterior descending (LAD) coronary artery was ligated near its origin with 6-0 prolene suture (Ethicon).

10. Cardiac Function Measurements

Transthoracic echocardiography was performed with a 6.0-15.0 MHz ultraband linear transducer connected to SONOS 5500 (Agilent Technologies, Andover, Mass., USA) as described {Kawamoto, 2003}. LVEDD, LVESD, FS was measured. All measurements represent the mean of at least 3 consecutive cardiac cycles. To evaluate the regional wall motion abnormality, wall motion score index (WMSI) was used {Oh J K, 1999}. LV wall motion analysis is based on grading contractility of individual segment. In this scoring system, higher scores indicate more severe wall motion abnormality (1, normal; 2, hypokinesis; 3, akinesis; 4, dyskinesis; 5, aneurysmal). A WMSI is derived by dividing the sum of wall motion scores by the number of visualized segments; it represents the extent of regional wall motion abnormalities. A normal WMSI is 1. To measure hemodynamic variables, 1.4 French high fidelity pressure transducer (Micro-tip catheter, Millar Instrument, Houston, Tex.) was introduced into the left ventricle via right carotid artery. After hemodynamic was stabilized, LVSP, LVEDP, the +dP/dt and −dP/dt were recorded using polygraph (Model 7P) (Grass Instrument, West Warwick, R.I., USA)[Kawamoto, 2001].

11. Immunofluorescent Histochemistry of Cardiac Tissues.

OCT embedded frozen sections were fixed in 4% PFA and used for follwowing immunohistochemistry. For identification of ECs, biotinylated isolectin B4 (1:200; Vector) was used as primary Abs followed by streptavidin-FITC(1:100) [Kawamoto, 2001]. SMCs were identified by mouse α-SMC Ab(Sigma, 1:200) followed by FITC-conjugated goat anti-mouse IgG. CMCs were identified by abs against cTnI and ANP followed by FITC-conjugated goat anti-rabbit IgG.

12. BrdU Immunohistochemistry and Determination of Apoptosis by TUNEL.

BrdU was detected with the sheep anti-BrdU antibody(1:50; Biodesign) followed by Streptavidin-FITC (1:100; Vector). Nuclear counterstaining was performed with DAPI. BrdU positive cells were counted in the peri-infarct area where the scar tissue comprises less than 20% of the visual field (×200 magnification).

In situ labeling of fragmented DNA was performed with terminal deoxynucleotidyltransferase (TdT)-mediated dUTP-biotin nick end labeling (TUNEL) method with the use of an in situ cell detection kit (Roche) as described [Fujio, 2000]. Two wk myocardial sections were used for TUNEL staining (n=4, each). Briefly, sections are treated with 20 μg of proteinase K. After washes, sections are incubated in a solution of TdT with fluorescein dUTP mixture. Sections are then counter-stained with DAPI for localization of nuclei. To determine the proportion of apoptotic nuclei within CMCs or ECs, tissue was counterstained with a mAb against α-sarcomeric actinin or ILB4. Tissue sections were examined microscopically under ×200 magnification, and a total of 8 random visual fields (total ˜10,000 nuclei) in the infarct/periinfarct area was examined. The percentage of apoptotic cells was termed the apoptotic index.

13. RT-PCR Analysis

RT-PCR was performed as described previously {Yoon, 2003}. Total RNA was extracted from cultured cells or from hearts using RNaqueous kit (Ambion, Austin, Tex., USA) according to the manufacturer's instruction. One microgram of total RNA were reverse transcribed using random hexamer and Moloney murine leukemia virus reverse transcriptase (Superscript II kit, Roche). The RT product was subjected to PCR using Advantage cDNA polymerase mix (Clontech) or Taq polymerase (Roche). For semiquantitative RT-PCR, quantification of mRNA expression of each gene was calculated based on the GAPDH expression. PCR primers and annealing temperature used is described in Table 1. RT-PCR products were analyzed by 1.5% agarose gel electrophoresis with a 100-bp ladder (Life Technologies) and quantified with the UV imager Eagle-Eye II(Stratagene).

14. Analysis of Cardiac Histology and Morphometry

Capillary density and CMC density was counted after staining of 4 wk samples with mAb against CD31 and H&E as described previously {Kawamoto, 2003; Kawamoto, 2001}. For counting, a total of 8 visual fields where a cross-section of capillaries and CMCs was clearly visible was randomly selected in the infarct/periinfarct area, and the number of capillaries or CMCs was counted under ×200 magnification (n=6, each group). To determine the fibrosis area, the average ratio of fibrosis area per LV area was measured for all histologic sections after staining with Masson's trichrome (Sigma) as described previously [Kawamoto, 2001].

15. Statistical analysis. Statistical analysis is performed by an unpaired Student's t-test for comparisons between two groups, ANOVA followed by Scheffe's post-hoc for more than two groups. P<0.05 is considered to denote statistical significance.

16. Polymerase Chain Reaction (PCR) Primers. Table 1 below shows oligonucleotide primers that were used as needed to conduct the experiments disclosed herein. “Product size” pertains to expected size (in basepairs) of amplified products.

TABLE 1
RT-PCR primer sequences and expected product size

| | | Product |
|---|---|---|
| Primers | Sequences | Size (bp) |
| Human | | |
| Oct4-f | 5′-GAG AAC AAT GAG AAC CTT CAG GAG A-3′ | 219 |
| Oct4-r | 5′-TTC TGG CGC CGG TTA CAG AAC CA-3′ | |
| CD34-f | 5′-ACC ACT TCC CTC ATC TCT CCT CCA A-3′ | 421 |
| CD34-r | 5′-AGG GTG AGG GAG GCA GAG ACA GAA A-3′ | |
| KDR-f | 5′-TGC AGG ACC AAG GAG ACT ATG T-3′ | 458 |
| KDR-r | 5′-TAG GAT GAT GAC AAG AAG TAG CC-3′ | |
| Tie2-f | 5′-ATC CCA TTT GCA AAG CTT CTG GCT GGC-3′ | 512 |
| Tie2-r | 5′-TGT GAA GCG TCT CAC AGG TCC AGG ATG-3′ | |
| CD31-f | 5′-AGG TCA GCA GCA TCG TGG TCA ACA T-3′ | 387 |
| CD31-r | 5′-GTG GGG TTG TCT TTG AAT ACC GCA G-3′ | |
| VE-cadherin-f | 5′-CTC TGC ATC CTC ACC ATC ACA G-3′ | 389 |
| VE-cadherin-r | 5′-TAG CCG TAG ATG TGC AGC GTG T-3′ | |
| SM1-f | 5′-TAA ACA CCT GCC CAT CTA CTC GG-3′ | 732 |
| SM1-r | 5′-ATC TCA TCA TCC TGG GCT GCT GG-3′ | |
| SM22α-f | 5′-CGG CTG GTG GAG TGG ATC ATA G-3′ | 489 |
| SM22α-r | 5′-CCC TCT GTT GCT GCC CAT CTG A-3′ | |
| PDGFRb-f | 5′-GCC TTA CCA CAT CCG CTC-3′ | 443 |
| PDGFRb-r | 5′-TCA CAC TCT TCC GTC ACA TTG C-3′ | |
| GATA4-f | 5′-AGA-CAT-CGC-ACT-GAC-TGA-GAA-C-3′ | 475 |
| GATA4-r | 5′-GAC-GGG-TCA-CTA-TCT-GTG-CAA-C-3′ | |
| Nkx2.5-f | 5′-CTT-CAA-GCC-AGA-GGC-CTA-CG-3′ | 233 |
| Nkx2.5-r | 5′-CCG-CCT-CTG-TCT-TCT-TCA-GC-3′ | |
| AFP-f | 5′-TGC AGC CAA AGT GAA GAG GGA AGA-3′ | 343 |
| AFP-r | 5′-CAT AGC CAG GAG CCC AAA GAA GAA-3′ | |
| Alb-f | 5′-TGC TTG AAT GTG CTG ATG ACA GGG-3′ | 181 |
| Alb-r | 5′-AAG GCA AGT CAG CAG GCA TCT CAT C-3′ | |
| CK18-f | 5′-GTA CTG GTC TCA GCA GAT TGA GGA G-3′ | 499 |
| CK18-r | 5′-GCT TCT GCT GGC TTA ATG CCT CAG A-3′ | |
| CK19-f | 5′-ATG GCC GAG CAG AAC CGG AA-3′ | 318 |
| CK19-r | 5′-CCA TGA GCC GCT GGT ACT CC-3′ | |
| GFAP-f | 5′-TCA TCG CTC AGG AGG TCC TT-3′ | 383 |
| GFAP-r | 5′-CTG TTG CCA GAG ATG GAG GTT-3′ | |
| MAP2-f | 5′-GAA GAC TCG CAT CCG AAT GG-3′ | 527 |
| MAP2-r | 5′-CGC AGG ATA GGA GGA AGA GAC T-3′ | |
| MBP-f | 5′-TTAGCT GAATTC GCG TGT GG-3′ | 374 |
| MBP-r | 5′-GAG GAAGTGAAT GAG CCG GTTA-3′ | |
| GAD-f | 5′-GCG CCA TAT CCA ACA GTG ACA G-3′ | 284 |
| GAD-r | 5′-GCC AGC AGT TGC ATT GAC ATA A-3′ | |
| Tau-f | 5′-GTA AAA GCA AAG ACG GGA CTG G-3′ | 512/612 |
| Tau-r | 5′-ATG ATG GAT GTT GCC TAA TGA G-3′ | |
| Rat | | |
| VEGF-f | 5′-GGA CCC TGA CTT TAC TGC TGT ACC-3′ | 434, 564, |
| VEGF-r | 5′-CCG AAA CCC TGA GGA GGC TCC-3′ | 631 |
| bFGF-f | 5′-TCT ACT GCA AGA ACG GCG GCT TCT T-3′ | 287 |
| bFGF-r | 5′-CAG TGC CAC ATA CCA ACT GGA GTA T-3′ | |
| PDGF-B-f | 5′-CCG AGG AGC TTT ATG AGA TGC TGA G-3′ | 479 |
| PDGF-B-r | 5′-AGC TGC CAC TGT CTC ACA CTT GCA T-3′ | |
| Nkx2.5-f | 5′-CAG TGG AGC TGG ACA AAG CC-3′ | 216 |
| Nkx2.5-r | 5′-TAG CGA CGG TTC TGG AAC CA-3′ | |
| GATA4-f | 5′-CTG TCA TCT CAC TAT GGG CA-3′ | 275 |
| GATA4-r | 5′-CCA AGT CCG AGC AGG AAT TT-3′ | |
| MEF2c-f | 5′-AGC AAG AAT ACG ATG CCA TC-3′ | |
| MEF2c-r | 5′-GAA GGG GTG GTG GTA CGG TC-3′ | 407, 311 |
| Ang-1-f | 5′-AGT CGG AGA TGG CCC AGA TAC AAC A-3′ | |
| Ang-1-r | 5′-TCC AGC AGT TGG ATT TCA AGA CGG G-3′ | 169 |
| Ang-2-f | 5′-TAC GTG CTG AAG ATC CAG CTG AAG G-3′ | |
| Ang-2-r | 5′-AGT TGG AAG GAC CAC ATG CGT CGA A-3′ | 259 |
| HGF-f | 5′-CCA ACA CAA ACA ACA GA GGG TGG A-3′ | |
| HGF-r | 5′-CGA CCA GGA ACA ATG ACA CCA AGA A-3′ | 593 |
| TGF-β-f | 5′-CAA CTA CTG CTT CAG CTC CAC AGA G-3′ | |
| TGF-β-r | 5′-AGG AGC GCA CGA TCA TGT TGG ACA A-3′ | 314 |
| GAPDH-f | 5′-TCG GTG TGA ACG GAT TTG GCC GTA T-3′ | |
| GAPDH-r | 5′-AGC CCT TCC ACG ATG CCA AAG TTG T-3′ | 505′ |

-f: forward primer, -r: reverse primer

Supplementary Information

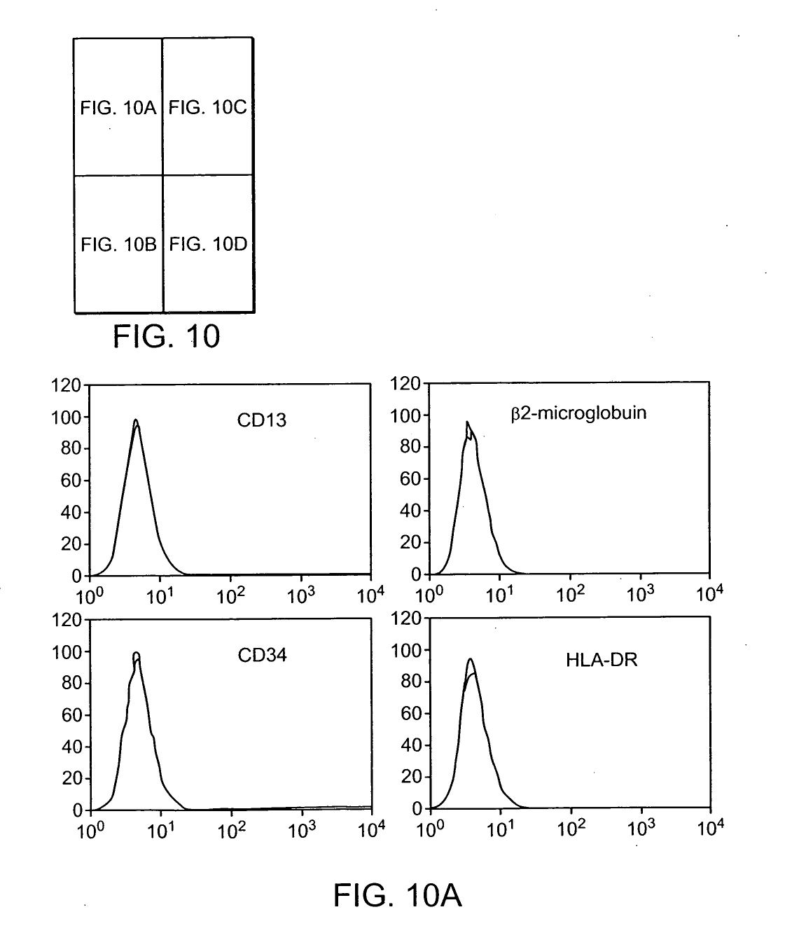

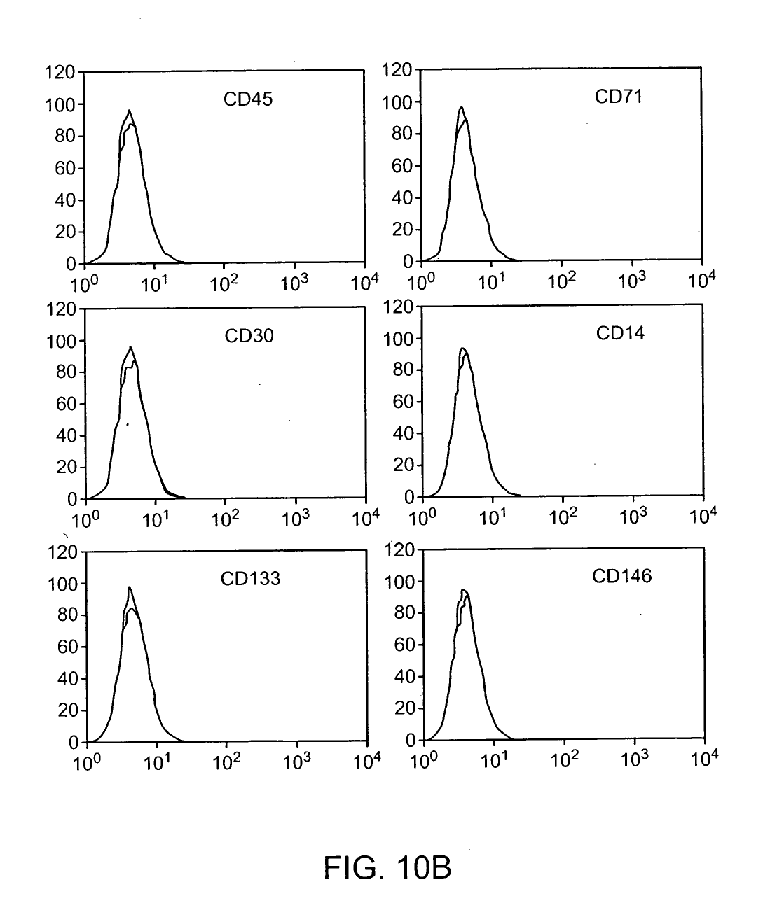

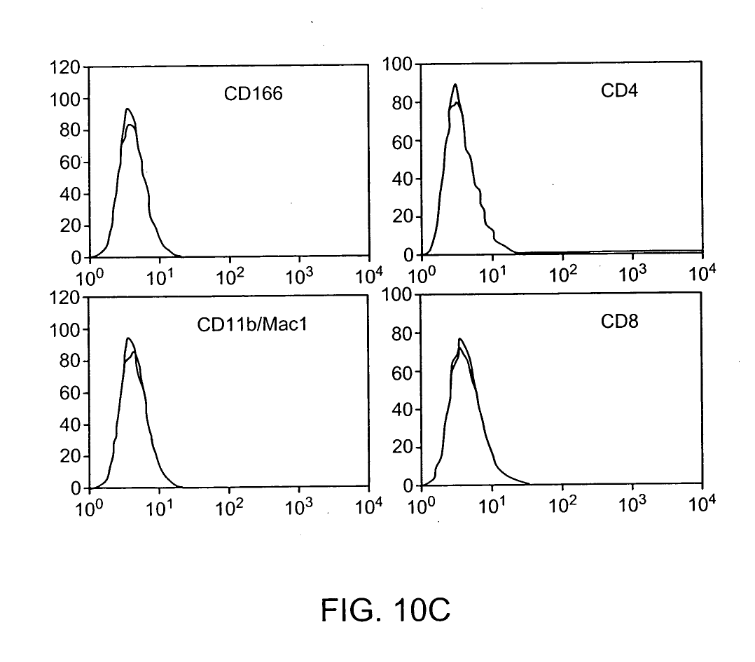

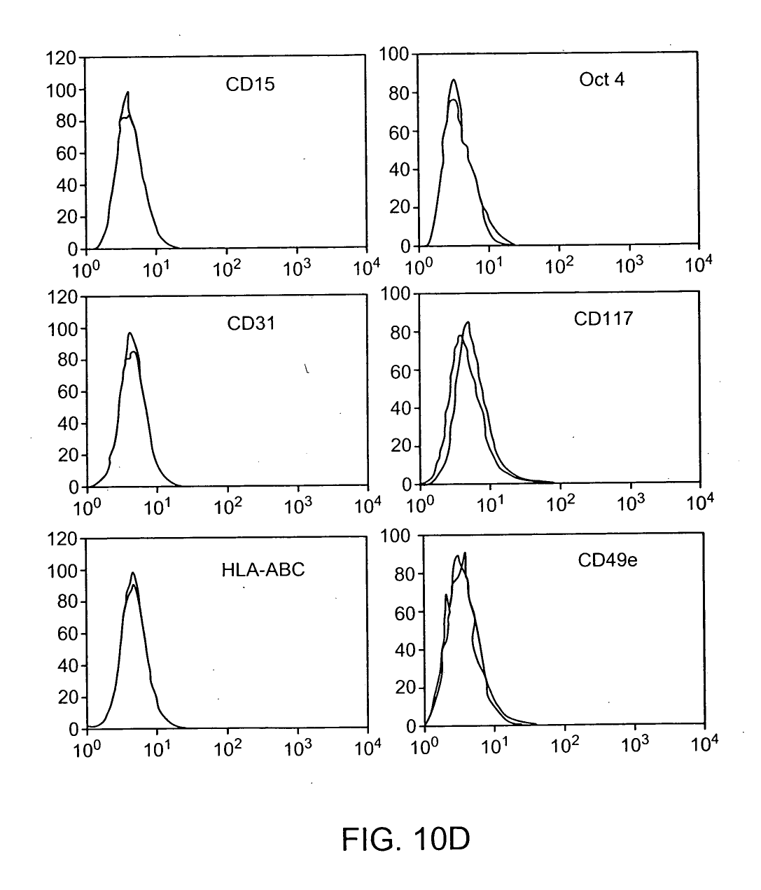

FIG. 10. FACS analysis using multiple surface epitopes demonstrated none to minimal expression (<1%) of CD117 in hBMSC. Major histocompatibility complex (MHC) class I(ABC) and II(DR) were negative and known HSC markers(CD34, CD133, Flk-1, Tie2) were not expressed.

The following references 1-80 are referred to throughout the instant disclosure and are incorporated herein by reference.

### REFERENCES

1. Braunwald, E. & Bristow, M. R. Congestive Heart Failure: Fifty Years of Progress. Circulation 102, 14IV-23 (2000).
2. Ho, K. K., Anderson, K. M., Kannel, W. B., Grossman, W. & Levy, D. Survival after the onset of congestive heart failure in Framingham Heart Study subjects. Circulation 88, 107-15 (1993).
3. Pfeffer, M. A. Left ventricular remodeling after acute myocardial infarction. Annu Rev Med 46, 455-66 (1995).
4. Chien, K. R. Stress pathways and heart failure. Cell 98, 555-8 (1999).
5. Kopen, G. C., Prockop, D. J. & Phinney, D. G. Marrow stromal cells migrate throughout forebrain and cerebellum, and they differentiate into astrocytes after injection into neonatal mouse brains. Proc Natl Acad Sci USA 96, 10711-6 (1999).
6. Brazelton, T. R., Rossi, F. M., Keshet, G. I. & Blau, H. M. From marrow to brain: expression of neuronal phenotypes in adult mice. Science 290, 1775-9 (2000).
7. Mezey, E., Chandross, K. J., Harta, G., Maki, R. A. & McKercher, S. R. Turning blood into brain: cells bearing neuronal antigens generated in vivo from bone marrow. Science 290, 1779-82 (2000).
8. Ferrari, G. et al. Muscle regeneration by bone marrow-derived myogenic progenitors. Science 279, 1528-30 (1998).
9. Gussoni, E. et al. Dystrophin expression in the mdx mouse restored by stem cell transplantation. Nature 401, 390-4 (1999).
10. Goodell, M. A. et al. Stem cell plasticity in muscle and bone marrow. Ann NY Acad Sci 938, 208-18; discussion 218-20. (2001).
11. Orlic, D. et al. Bone marrow cells regenerate infarcted myocardium. Nature 410, 701-5. (2001).
12. Jackson, K. A. et al. Regeneration of ischemic cardiac muscle and vascular endothelium by adult stem cells. J Clin Invest 107, 1395-402. (2001).
13. Asahara, T. et al. Bone marrow origin of endothelial progenitor cells responsible for postnatal vasculogenesis in physiological and pathological neovascularization. Circ Res 85, 221-8. (1999).
14. Lin, Y., Weisdorf, D. J., Solovey, A. & Hebbel, R. P. Origins of circulating endothelial cells and endothelial outgrowth from blood. J Clin Invest 105, 71-7 (2000).
15. Lagasse, E. et al. Purified hematopoietic stem cells can differentiate into hepatocytes in vivo. Nat Med 6, 1229-34 (2000).
16. Petersen, B. E. et al. Bone marrow as a potential source of hepatic oval cells. Science 284, 1168-70 (1999).
17. Krause, D. S. et al. Multi-organ, multi-lineage engraftmentby a single bone marrow-derived stem cell. Cell 105, 369-77 (2001).
18. Reyes, M. et al. Origin of endothelial progenitors in human postnatal bone marrow. J. Clin Invest 109, 337-46 (2002).
19. Reyes, M. et al. Purification and ex vivo expansion of postnatal human marrow mesodermal progenitor cells. Blood 98, 2615-25 (2001).
20. Jiang, Y. et al. Pluripotency of mesenchymal stem cells derived from adult marrow. Nature 418, 41-9 (2002).
21. Schwartz, R. E. et al. Multipotent adult progenitor cells from bone marrow differentiate into functional hepatocyte-like cells. J Clin Invest 109, 1291-302 (2002).
22. Kawamoto, A. et al. Therapeutic potential of ex vivo expanded endothelial progenitor cells for myocardial ischemia. Circulation 103, 634-7. (2001).
23. Kocher, A. A. et al. Neovascularization of ischemic myocardium by human bone-marrow-derived angioblasts prevents cardiomyocyte apoptosis, reduces remodeling and improves cardiac function. Nat Med 7, 430-6. (2001).
24. Kawamoto, A. et al. Intramyocardial transplantation of autologous endothelial progenitor cells for therapeutic neovascularization of myocardial ischemia. Circulation 107, 461-8 (2003).
25. Prockop, D. J. Marrow stromal cells as stem cells for nonhematopoietic tissues. Science 276, 71-4 (1997).
26. Pittenger, M. F. et al. Multilineage potential of adult human mesenchymal stem cells. Science 284, 143-7 (1999).
27. Horwitz, E. M. & Keating, A. Nonhematopoietic mesenchymal stem cells: what are they? Cytotherapy 2, 387-8 (2000).
28. Taylor, S. M. & Jones, P. A. Multiple new phenotypes induced in 10T1/2 and 3T3 cells treated with 5-azacytidine. Cell 17, 771-9 (1979).
29. Makino, S. et al. Cardiomyocytes can be generated from marrow stromal cells in vitro. J Clin Invest 103, 697-705 (1999).
30. Hakuno, D. et al. Bone Marrow-Derived Regenerated Cardiomyocytes (CMG Cells) Express Functional Adrenergic and Muscarinic Receptors. Circulation 105, 380-6. (2002).
31. Tomita, S. et al. Autologous transplantation of bone marrow cells improves damaged heart function. Circulation 100, II247-56. (1999).
32. Wang, J. S. et al. Marrow stromal cells for cellular cardiomyoplasty: feasibility and potential clinical advantages. J Thorac Cardiovasc Surg 120, 999-1005 (2000).
33. Shake, J. G. et al. Mesenchymal stem cell implantation in a swine myocardial infarct model: engraftment and ftunctional effects. Ann Thorac Surg 73, 1919-25; discussion 1926 (2002).
34. Toma, C., Pittenger, M. F., Cahill, K. S., Byrne, B. J. & Kessler, P. D. Human mesenchymal stem cells differentiate to a cardiomyocyte phenotype in the adult murine heart. Circulation 105, 93-8 (2002).
35. Orlic, D. et al. Transplanted adult bone marrow cells repair myocardial infarcts in mice. Ann NY Acad Sci 938, 221-9; discussion 229-30. (2001).
36. Hellstrom, M., Kal n, M., Lindahl, P., Abramsson, A. & Betsholtz, C. Role of PDGF-B and PDGFR-beta in recruitment of vascular smooth muscle cells and pericytes during embryonic blood vessel formation in the mouse. Development 126, 3047-55 (1999).
37. Yamashita, J. et al. Flk1-positive cells derived from embryonic stem cells serve as vascular progenitors. Nature 408, 92-6 (2000).
38. Palmer, T. D., Markakis, E. A., Willhoite, A. R., Safar, F. & Gage, F. H. Fibroblast growth factor-2 activates a latent neurogenic program in neural stem cells from diverse regions of the adult CNS. J Neurosci 19, 8487-97 (1999).
39. Sanchez-Ramos, J. et al. Adult bone marrow stromal cells differentiate into neural cells in vitro. Exp Neurol 164, 247-56 (2000).
40. Shen, C. N., Slack, J. M. & Tosh, D. Molecular basis of transdifferentiation of pancreas to liver. Nat Cell Biol 2, 879-87 (2000).
41. Hamazaki, T. et al. Hepatic maturation in differentiating embryonic stem cells in vitro. FEBS Lett 497, 15-9 (2001).
42. Oh, S. H. et al. Hepatocyte growth factor induces differentiation of adult rat bone marrow cells into a hepatocyte lineage in vitro. Biochem Biophys Res Commun 279, 500-4 (2000).
43. Condorelli, G. et al. Cardiomyocytes induce endothelial cells to trans-differentiate into cardiac muscle: implications for myocardium regeneration. Proc Natl Acad Sci USA 98, 10733-8 (2001).
44. Srivastava, D. & Olson, E. N. A genetic blueprint for cardiac development. Nature 407, 221-6 (2000).
45. Bruneau, B. G. Transcriptional regulation of vertebrate cardiac morphogenesis. Circ Res 90, 509-19 (2002).
46. Ying, Q. L., Nichols, J., Evans, E. P. & Smith, A. G. Changing potency by spontaneous fusion. Nature 416, 545-8 (2002).
47. Terada, N. et al. Bone marrow cells adopt the phenotype of other cells by spontaneous cell fusion. Nature 416, 542-5 (2002).
48. Wang, X. et al. Cell fusion is the principal source of bone-marrow-derived hepatocytes. Nature 422, 897-901 (2003).
49. Vassilopoulos, G., Wang, P. R. & Russell, D. W. Transplanted bone marrow regenerates liver by cell fusion. Nature 422, 901-4 (2003).
50. Carmeliet, P. Mechanisms of angiogenesis and arteriogenesis. Nat Med 6, 389-95 (2000).
51. Thursdon, G. et al. Angiopoietin-1 protects the adult vasculature against plasma leakage. Nature Medicine 6, 460-463 (2000).
52. Asahara, T. et al. Tie2 receptor ligands, angiopoietin-1 and angiopoietin-2, modulate VEGF-induced postnatal neovascularization. Circ Res 83, 233-40. (1998).
53. Bussolino, F. et al. Hepatocyte growth factor is a potent angiogenic factor which stimulates endothelial cell motility and growth. J Cell Biol 119, 629-41 (1992).
54. Chelboun, J. O., Martins, R. N., Mitchell, C. A. & Chirila, T. V. bFGF enhances the development of collateral circulation after acute arterial occlusion. Biochemical and Biophysical Research Communications 185, 510-516 (1992).
55. Folkman, J. & D'Amore, P. A. Blood vessel formation: what is its molecular basis? Cell 87, 1153-5 (1996).
56. Vinals, F. & Pouyssegur, J. Transforming growth factor betal (TGF-betal) promotes endothelial cell survival during in vitro angiogenesis via an autocrine mechanism implicating TGF-alpha signaling. Mol Cell Biol 21, 7218-30 (2001).
57. Barry, F. P., Boynton, R. E., Haynesworth, S., Murphy, J. M. & Zaia, J. The monoclonal antibody SH-2, raised against human mesenchymal stem cells, recognizes an epitope on endoglin (CD105). Biochen Biophys Res Commun 265, 134-9 (1999).
58. Barry, F., Boynton, R., Murphy, M., Haynesworth, S. & Zaia, J. The SH-3 and SH-4 antibodies recognize distinct epitopes on CD73 from human mesenchymal stem cells. Biochem Biophys Res Commun 289, 519-24 (2001).
59. Colter, D. C., Sekiya, I. & Prockop, D. J. Identification of a subpopulation of rapidly self-renewing and multipotential adult stem cells in colonies of human marrow stromal cells. Proc Natl Acad Sci USA 98, 7841-5 (2001).
60. Yoon Y S, M. T., Tkebuchava T, Kawamoto A, Wecker A, Weber A, Hanley A, Bosch-Marce M, Heyd L, Kearney M, Asahara T, Losordo D W. Clonally Expanded Bone Marrow (BM) Derived Stem Cells Differentiate into Multiple Lineages In Vitro and Can Attenuate Myocardial Dysfunction Post Myocardial Infarction (MI). Circulation 106 (2002).
61. Vignery, A. Osteoclasts and giant cells: macrophage-macrophage fusion mechanism. Int J Exp Pathol 81, 291-304 (2000).
62. Mintz, B. & Baker, W. W. Normal mammalian muscle differentiation and gene control of isocitrate dehydrogenase synthesis. Proc Natl Acad Sci USA 58, 592-8 (1967).
63. Kerbel, R. S., Lagarde, A. E., Dennis, J. W. & Donaghue, T. P. Spontaneous fusion in vivo between normal host and tumor cells: possible contribution to tumor progression and metastasis studied with a lectin-resistant mutant tumor. Mol Cell Biol 3, 523-38 (1983).
64. Olivetti, G., Capasso, J. M., Meggs, L. G., Sonnenblick, E. H. & Anversa, P. Cellular basis of chronic ventricular remodeling after myocardial infarction in rats. Circ Res 68, 856-69 (1991).
65. Cheng, W. et al. Programmed myocyte cell death affects the viable myocardium after infarction in rats. Exp Cell Res 226, 316-27 (1996).
66. Yancopoulos, G. D. et al. Vascular-specific growth factors and blood vessel formation. Nature 407, 242-8 (2000).
67. Darland, D. C. & D'Amore, P. A. Blood vessel maturation: vascular development comes of age. J Clin Invest 103, 157-8 (1999).
68. Ferrara, N. & Alitalo, K. Clinical applications of angiogenic growth factors and their inhibitors. Nat Med 5, 1359-64 (1999).
69. Kamihata, H. et al. Implantation of bone marrow mononuclear cells into ischemic myocardium enhances collateral perfusion and regional function via side supply of angioblasts, angiogenic ligands, and cytokines. Circulation 104, 1046-52. (2001).
70. Giordano, F. J. et al. A cardiac myocyte vascular endothelial growth factor paracrine pathway is required to maintain cardiac function. Proc Natl Acad Sci USA 98, 5780-5 (2001).
71. Kitta, K. et al. Hepatocyte growth factor induces GATA-4 phosphorylation and cell survival in cardiac muscle cells. J Biol Chem 278, 4705-12 (2003).
72. Min, J. Y. et al. Transplantation of embryonic stem cells improves cardiac function in postinfarcted rats. J Appl Physiol 92, 288-96. (2002).
73. Kehat, I. et al. Human embryonic stem cells can differentiate into myocytes with structural and functional properties of cardiomyocytes. J Clin Invest 108, 407-14. (2001).
74. Kalka, C. et al. Transplantation of ex vivo expanded endothelial progenitor cells for therapeutic neovascularization. Proc Natl Acad Sci USA 97, 3422-7. (2000).
75. De Luca, A. et al. Characterization of caveolae from rat heart: localization of postreceptor signal transduction molecules and their rearrangement after norepinephrine stimulation. J Cell Biochem 77, 529-39 (2000).
76. McEwan, P. E., Gray, G. A., Sherry, L., Webb, D. J. & Kenyon, C. J. Differential effects of angiotensin II on cardiac cell proliferation and intramyocardial perivascular fibrosis in vivo. Circulation 98, 2765-73 (1998).
77. Oh J K, S. J., Tajik A J. Assessment of ventricular systolic function. in The Echo Manual (ed. Oh J K, S. J., Tajik A J) 37-43 (Lippincott-Raven, Philadelphia, 1999).
78. Isner, J. M., Kalka, C., Kawamoto, A. & Asahara, T. Bone marrow as a source of endothelial cells for natural and iatrogenic vascular repair. Ann N YAcad Sci 953, 75-84. (2001).
79. Fujio, Y., Nguyen, T., Wencker, D., Kitsis, R. N. & Walsh, K. Akt promotes survival of cardiomyocytes in vitro and protects against ischemia-reperfusion injury in mouse heart. Circulation 101, 660-7 (2000).
80. Yoon, Y. S. et al. VEGF-C gene therapy augments postnatal lymphangiogenesis and ameliorates secondary lymphedema. J Clin Invest 111, 717-25 (2003).

While the invention has been described with reference to specific embodiments, modifications and variations of the invention may be constructed without departing from the scope of the invention, which is defined in the following claims. All references are incorporated herein by reference.

1. An isolated bone marrow stem cell having undetectable or low levels of at least one of and preferably all of the following cell markers: CD90, CD117, CD34, CD113, FLK-1, tie-2, Oct 4, GATA-4, NKx2.5, Rex-1, CD105, CD117, CD133, MHC class I receptor and MHC class II receptor as determined by standard cell marker detection assay.

2. The isolated bone marrow stem cell of claim 1, wherein the cells are essentially spherical as determined by inspection and have a diameter of less than about 25 to 35 micrometers, preferably less than about 15 micrometers.

3. The isolated bone marrow stem cell of claim 1, wherein the cells have a mean telomere restriction fragment length (TRF) less than about 30 to 40 kilobases.

4. The isolated bone marrow stem cell of claim 1, wherein the cells are essentially euploid.

5. The isolated bone marrow stem cell of claim 1, wherein euploidy of the cells is essentially maintained for at least about 10 passages in cell culture, preferably between from about 20 to about 200 passages.

6. The isolated bone marrow stem cell of claim 1, wherein the cells form endothelial cells (ECs) after contact with EC promoting conditions as determined by a standard EC differentiation assay.

7. The isolated bone marrow stem cell of claim 1, wherein the EC promoting conditions include contact with a vascular endothelial growth factor (VEGF).

8. The isolated bone marrow stem cell of claim 1, wherein the cells form smooth muscle cells (SMCs) after contact with SMC promoting conditions as determined by a standard SMC differentiation assay.

9. The isolated bone marrow stem cell of claim 8, wherein the SMC promoting conditions include contact with a platelet derived growth factor (PDGF).

10. The isolated bone marrow stem cell of claim 1, wherein the cells form neuronal cells after contact with neuronal cell promoting conditions as determined by a standard neuronal cell differentiation assay.

11. The isolated bone marrow stem cell of claim 10, wherein the neuronal cell promoting conditions include contact with hepatocyte growth factor (HGF).

12. The isolated bone marrow stem cell of claim 11, wherein the neuronal cell promoting conditions include further contact with fibroblast growth factor 4 (FGF-4).

13. The isolated bone marrow stem cell of claim 12, wherein the neuronal cell promoting conditions include further contact with either DMSO or a pharmaceutically acceptable butyrate salt.

14. The isolated bone marrow stem cell of claim 1, wherein the cells form cardiomyocytes after contact with accessory cardiomyocytes.

15. The isolated bone marrow stem cell of claim 14, wherein at least some of the formation is associated with cell fusion between the cells and the accessory cardiomyocytes.

16. The isolated bone marrow stem cell of claim 15, wherein the accessory cardiomyocytes are being maintained in vitro, ex vivo or in vivo.

17. A graft comprising the isolated bone marrow stem cells of claim 1.

18. The graft of claim 17, wherein the graft further comprises cardiomyocytes produced from the isolated bone marrow cells.

19. The graft of claim 17, wherein the graft further comprises ECs produced by the isolated bone marrow cells.

20. The graft of claim 17, wherein the graft further comprises SMCs produced by the isolated bone marrow cells.

21. The graft of claim 17, the graft further comprising cells from a recipient, wherein the recipient cells and the bone marrow stem cells are mammalian.

22. The graft of claim 21, wherein the recipient cells and the isolated bone marrow stem cells are allogenic, autologous or syngeneic.

23. The graft of claim 17, wherein the graft is maintained in vivo or in vitro.

24. The graft of claim 18, wherein the cardiomyocytes produced from the bone marrow cells express CMC specific protein (cTnI).

25. The graft of claim 19, wherein the ECs produced from the bone marrow cells express ILB-4.

26. The graft of claim 20, wherein the SMCs produced from the bone marrow cells express α-SMA.

27. A cell culture, tissue or organ comprising the graft of claim 17.

28. A method for preventing, treating or reducing the severity of a heart disorder, the method comprising administering to a mammal in need of treatment the isolated bone marrow cell of claim 1 and the graft of claim 17, wherein the administration is sufficient to prevent, treat or reduce the severity of the disorder in the mammal.

29. The method of claim 28, wherein the method further comprises incubating the cells or graft in the mammal for at least about a week.

30. The method of claim 29, wherein the incubation in the mammal is between from about two to eight weeks.

31. The method of claim 28, wherein the method further comprising administering to the mammal at least one angiogenic factor.

32. The method of claim 28, wherein the method further comprises administering at least one nucleic acid encoding at least one angiogenic factor or functional fragment thereof.

33. The method of claim 28, wherein the method further comprises administering to the mammal endothelial progenitor cells (EPCs).

34. The method of claim 33, wherein the method further comprises isolating the EPCs from the mammal and contacting the EPCs with at least one angiogenic factor ex vivo.

35. The method of claim 28, wherein the heart disorder is one or more of congestive heart failure (CHF), ischemic cardiomyopathy, myocardial ischemia, or an infarct.

36. The method of claim 28, wherein the method further comprises monitoring cardiac function in the mammal.

37. The method of claim 36, wherein the monitored cardiac function is at least one of echocardiography, ventricular end-diastolic dimension (LVEDD), end-systolic dimension (LVESD), fractional shortening (FS), wall motion score index (WMSI) and LV systolic pressure (LVSP).

38. A pharmaceutical product for preventing, treating or reducing the severity of a heart disorder, the product comprising at least one of the following components: the isolated bone marrow cells of claim 1 and optionally directions for isolating the cells from a mammal; the graft of claim 17 and optionally directions for preparing, maintaining and/or using the graft; the cell culture, tissue or organ of claim 27 and optionally directions for preparing same.

39. The pharmaceutical product of claim 38, wherein the product further comprises at least one angiogenic factor or functional fragment thereof.

40. The pharmaceutical product of claim 38, wherein the product further comprises at least one nucleic acid encoding at least one angiogenic factor or functional fragment thereof.

41. A population of isolated bone marrow cells obtained by the following process:
a) collecting bone marrow cells from a mammal which cells have a size of less than about 100 microns, preferably less than about 50 microns, more preferably about 40 microns or less,
b) culturing (expanding) the collected cells in medium under conditions that select for adherent cells,
c) selecting the adherent cells and expanding those cells in medium to semi-confluency,
d) serially diluting the cultured cells into chambers with conditioned medium, the dilution being sufficient to produce a density of less than about 1 cell per chamber to make clonal isolates of the expanded cells; and
e) culturing (expanding) each of the clonal isolates and selecting chambers having expanded cells to make the population of isolated bone marrow cells.

42. The population of isolated bone marrow cells of claim 40, wherein the process further comprises collecting cells that do not express detectable levels of at least one of the following markers: CD90, CD117; CD34, CD113, FLK-1, tie-2, Oct 4, GATA-4, NKx2.5, Rex-1, CD105, CD117, CD133, MHC class I receptor and MHC class II receptor.

43. A method of making the isolated bone marrow cells of claim 1, the method comprising:
a) collecting bone marrow cells from a mammal which cells have a size of less than about 100 microns, preferably less than about 50 microns, more preferably about 40 microns or less,
b) culturing (expanding) the collected cells in medium under conditions that select for adherent cells,
c) selecting the adherent cells and expanding those cells in medium to semi-confluency,
d) serially diluting the cultured cells into chambers with conditioned medium, the dilution being sufficient to produce a density of less than about 1 cell per chamber to make clonal isolates of the expanded cells; and
e) culturing (expanding) each of the clonal isolates and selecting chambers having expanded cells to make the population of isolated bone marrow cells.

44. The method of claim 43, further comprises collecting cells that do not express detectable levels of at least one of the following markers: CD90, CD117, CD34, CD113, FLK-1, tie-2, Oct 4, GATA-4, NKx2.5, Rex-1, CD105, CD117, CD133, MHC class I receptor and MHC class II receptor.
